(19) United States
(12) Patent Application Publication
(10) Pub. No.: US 2017/0029777 A1
(43) Pub. Date: Feb. 2, 2017

(54) METHODS OF EXPANDING EX VIVO NATURAL KILLER T (NKT) CELLS AND THERAPEUTIC USES THEREOF

(71) Applicant: St. Jude Children's Research Hospital, Inc., Memphis, TN (US)

(72) Inventor: Asha PILLAI, Arlington, TN (US)

(73) Assignee: ST. JUDE CHILDREN'S RESEARCH HOSPITAL, INC., Memphis, TN (US)

(21) Appl. No.: 15/114,259

(22) PCT Filed: Jan. 23, 2015
(86) PCT No.: PCT/US2015/012580

§ 371 Date: Jul. 26, 2016

Related U.S. Application Data
(60) Provisional application No. 61/931,744, filed on Jan. 27, 2014.

(51) Int. Cl. C12N 5/0783; A61K 39/00; A61K 35/17

(52) U.S. Cl. CPC C12N 5/0646; A61K 35/17; A61K 39/001; A61K 2039/5158; A61K 2039/577; C12N 2500/36; C12N 2501/2302; C12N 2501/2307; C12N 2501/515; C12N 2501/599; C12N 2506/11

(57) ABSTRACT

The present invention is directed to novel methods of producing ex vivo natural killer T (NKT) cells, and therapeutic uses thereof for treatment of certain conditions including cancer, autoimmunity, inflammatory disorders, allergic disorders, tissue transplant-related disorders, and infections.

### CROSS-REFERENCE TO RELATED APPLICATION

This application claims priority to U.S. Provisional Application No. 61/931,744, filed on Jan. 27, 2014, the disclosure of which is herein incorporated by reference in its entirety.

### FIELD OF THE INVENTION

The present invention is directed to novel methods of producing ex vivo natural killer T (NKT) cells and therapeutic uses thereof for treatment of certain conditions including cancer, autoimmunity, inflammatory disorders, tissue transplant-related disorders, and infections.

### BACKGROUND OF THE INVENTION

Natural killer (NK) cells are lymphocytes that function at the interface between innate and adaptive immunity. NK cells contribute directly to immune defense through their effector functions, such as cytotoxicity and cytokine secretion, and indirectly by regulating antigen-presenting cells (APCs) and the adaptive responses of T cells. NK cells have the capacity to distinguish diseased cells from healthy cells, to mount powerful antiviral responses, and to maintain the pool of long-lived cells that expands during a response.

Natural killer T (NKT) cells represent a small population of T lymphocytes defined by the expression of both αβ T-cell receptors (TCR) and some lineage markers of NK cells. There are a number of subtypes of NKT cells, which can be determined through their T cell receptor (TCR) usage, cytokine production, expression of specific surface molecules and reactivity. The most extensively characterized subtype of NKT cells are the so-called type I or invariant natural killer T cell (iNKT cells) (Matsuda et al, Curr Opin Immunol, 20: 358-68, 2008). The TCR repertoire expressed by iNKT cells is invariant—i.e., a canonical α-chain (Vα24-Jα18 in humans; Vα14-Jα18 in mice) associated with a limited spectrum of β chains (Vβ11 in humans; Vβ8.2, Vβ2, Vβ7 in mice). This is in contrast to the polymorphic TCRs expressed by so-called nonclassical or noninvariant type II NKT cells (Porcelli et al, J Exp Med, 178: 1-16, 1993).

Although iNKT cells represent a relatively low frequency of peripheral blood T cells in humans, their limited TCR diversity means that they respond at high frequency following activation. iNKT cells are uniquely positioned to shape adaptive immune responses and have been demonstrated to play a modulatory role in a wide variety of diseases such as cancer, autoimmunity, inflammatory disorders, tissue transplant-related disorders, and infection (Terabe & Berzofsky, Ch. 8, Adv Cancer Res, 101: 277-348, 2008; Wu & van Kaer, Curr Mol Med, 9: 4-14, 2009; Tessmer et al, Expert Opin Ther Targets, 13: 153-162, 2009). For example, mice deficient in NKT cells are susceptible to the development of chemically induced tumors, whereas wild-type mice are protected (Guerra et al, Immunity 28: 571-80, 2008). These experimental findings correlate with clinical data showing that patients with advanced cancer have decreased iNKT cell numbers in peripheral blood (Gilfillan et al, J Exp Med, 205: 2965-73, 2008).

iNKT cells constitute <0.1% of peripheral blood and <1% of bone marrow T cells in humans, but despite their relative scarcity, they exert potent immune regulation via production of IL-2, Th1-type (IFN-γ, TNF-α), Th2-type (IL-4, IL-13), IL-10, and IL-17 cytokines. (Lee et al, J Exp Med, 2002; 195: 637-641; Bendelac et al, Annu Rev Immunol, 2007; 178: 58-66; Burrows et al, Nat Immunol, 2009; 10(7): 669-71). iNKT cells are characterized by a highly restricted (invariant) T-cell receptor (TCR)-Vα chain (Vα24 in humans). Their TCR is unique in that it recognizes altered glycolipids of cell membranes presented in context of a ubiquitous HLA-like molecule, CD1d. (Zajonc & Kronenberg, Immunol Rev, 2009; 230 (1): 188-200). CD1d is expressed at high levels on many epithelial and hematopoietic tissues and on numerous tumor targets, and is known to specifically bind only the iNKT TCR. (Borg et al, Nature, 2007, 448: 44-49).

Like NK cells, iNKT cells play a major role in tumor immunosurveillance, via direct cytotoxicity mediated through perforin/Granzyme B, Fas/FasL, and TRAIL pathways. (Brutkiewicz & Sriram, Crit Rev Oncol Hematol, 2002; 41: 287-298; Smyth et al, J. Exp. Med. 2002; 191: 661-8; Wilson & Delovitch, Nat Rev Immunol, 2003; 3: 211-222; Molling et al, Clinical Immunology, 2008; 129: 182-194; Smyth et al, J Exp Med, 2005; 201 (12):1973-1985; Godfrey et al, Nat Rev Immunol, 2004, 4: 231-237). In mice, iNKT cells protect against GVHD, while enhancing cytotoxicity of many cell populations including NK cells (FIG. 5). Unlike NK cells, iNKT cells are not known to be inhibited by ligands such as Class I MHC, making them very useful adjuncts in settings of tumor escape from NK cytotoxicity via Class I upregulation (FIG. 5). (Brutkiewicz & Sriram, Crit Rev Oncol Hematol, 2002; 41: 287-298; Smyth et al, J Exp Med 2002; 191: 661-8; Wilson & Delovitch, Nat Rev Immunol, 2003; 3: 211-222; Molling et al, Clinical Immunology, 2008; 129: 182-194; Smyth et al, J Exp Med, 2005; 201 (12):1973-1985; Godfrey et al, Nat Rev Immunol, 2004, 4: 231-237).

Further evidence supporting an important role for iNKT cells in antitumor immunity is provided in studies using Jα18 gene-targeted knockout mice that exclusively lack iNKT cells (Smyth et al, J Exp Med, 191: 661-668, 2000). For example, iNKT-deficient mice exhibited significantly increased susceptibility to methylcholanthrene-induced sarcomas and melanoma tumors, an effect reversed by the administration of liver-derived iNKT cells during the early stages of tumor growth (Crowe et al, J Exp Med, 196: 119-127, 2002).

At least one contribution of iNKT cells to antitumor immunity occurs indirectly via the activation of iNKT cells by DCs. Activated iNKT cells can initiate a series of cytokine cascades—including production of interferon gamma (IFN-γ)—that helps boost the priming phase of the antitumor immune response (Terabe &. Berzofsky, Ch 8, Adv Cancer Res, 101: 277-348, 2008). IFN-γ production by iNKT cells, as well as NK cells and CD8+ effectors, has been shown to be important in tumor rejection (Smyth et al, Blood, 99: 1259-1266, 2002). The underlying mechanisms are well characterized (Uemura et al, J Imm, 183: 201-208, 2009).

Further, iNKT cells have been shown to specifically target the killing of CD1d-positive tumor-associated macrophages (TAMs), a highly plastic subset of inflammatory cells derived from circulating monocytes that perform immunosuppressive functions (Sica & Bronte, J Clin Invest, 117: 1155-1166, 2007). TAMs are known to be a major producer of interleukin-6 (IL-6) that promotes proliferation of many solid tumors, including neuroblastomas and breast and prostate carcinomas (Song et al., J Clin Invest, 119: 1524-1536, 2009; Hong et al, Cancer, 110: 1911-1928, 2007). Direct CD1d-dependent cytotoxic activity of iNKT cells against TAMs suggests that important alternative indirect pathways exist by which iNKT cells can mediate antitumor immunity, especially against solid tumors that do not express CD1d.

In humans, iNKT cells are home to neuroblastoma cells (Metelitsa et al, J Exp Med 2004; 199 (9):1213-1221) and B cell targets (Wilson & Delovitch, Nat Rev Immunol 2003; 3: 211-222; Molling et al, Clinical Immunology, 2008; 129: 182-194) both of which express high levels of CD1d. iNKT cell cytokines may increase NK cytotoxicity. IFN-γ enhances NK cell proliferation and direct cytotoxicity, whereas IL-10 potently increases TIA-1, a molecule within NK cytotoxic granules which has direct DNA cleavage effects (Tian et al, Cell, 1991; 67 (3): 629-39) and can regulate mRNA splicing in NK cell targets, favoring expression of membrane-bound Fas on targets. (Izquierdo et al, Mol Cell, 2005; 19 (4): 475-84). IL-10 further enhances tumor target susceptibility to NK lysis by inducing tumor downregulation of Class I MHC, a major inhibitory ligand for NK cells. (Kundu & Fulton, Cell Immunol, 1997; 180:55-61).

Evidence supporting an important role for iNKT cells in the treatment of inflammatory diseases and/or autoimmune diseases comes from studies using murine autoimmune disease models. For example, in mouse models of type I diabetes (M. Falcone et al, J Immunol, 172: 5908-5916, 2004; Mizuno et al, J Autoimmun, 23: 293-300, 2004), rheumatoid arthritis (Kaieda et al, Arthritis and Rheumatism, 56: 1836-1845, 2007; Miellot-Gafsou et al, Immunology, 130: 296-306, 2010), autoimmune colitis (Crohn's disease and ulcerative colitis models DSS-induced colitis and autoimmune T cell-mediated colitis; Geremia et al., Autoimmun Rev. 13(1):3-10, 2014 doi: 10.1016/j.autrev.2013.06.004. Epub 2013 Jun. 15. Katsurada et al., PLoS One, 7(9):e44113, 2012; Fuss and Strober, Mucosal Immunol., 1 Suppl 1:S31-3, 2008), and experimental autoimmune encephalitis (EAE) (van de Keere & Tonegawa, J Exp Med, 188: 1875-1882, 1998; Singh et al, J Exp Med, 194:1801-1811, 2001; Miyamoto et al, Nature, 413: 531-534, 2001), iNKT cells played key roles in establishing immune tolerance and preventing autoimmune pathology.

Evidence supporting an important role for iNKT cells in the treatment of diabetes comes from studies using non-obese diabetic (NOD) mice that develop a spontaneous form of type 1 diabetes (T1D) mediated by autoreactive T cells, in which iNKT cells can alter the kinetics of disease onset and severity of disease. In this model, such mice have been found to contain reduced numbers of NKT cells and either activation or increasing the number of iNKT cells in NOD mice affords a degree of protection from T1D (Baxter et al, Diabetes, 46:572-82, 1997).

iNKT cells are also activated and participate in responses to transplanted tissue. Without subscribing exclusively to any one theory, evidence supporting an important role for iNKT cells in transplantation-related disorders is hereby incorporated. For example, iNKT cells have been shown to infiltrate both cardiac and skin allografts prior to rejection and have been found in expanded numbers in peripheral lymphoid tissue following transplantation (Maier et al, Nat Med, 7: 557-62, 2001; Oh et al, J Immunol, 174: 2030-6, 2005; Jiang et al, J Immunol, 175: 2051-5, 2005). iNKT cells are not only activated, but also influence the ensuing immune response (Jukes et al, Transplantation, 84: 679-81, 2007). For example, it has been found consistently that animals deficient in either total NKT cells or iNKT cells are resistant to the induction of tolerance by co-stimulatory/co-receptor molecule blockade (Seino et al, Proc Natl Acad Sci USA, 98: 2577-81, 2001; Jiang et al, J Immunol, 175: 2051-5, 2005; Jiang et al, Am J Transplant, 7: 1482-90, 2007). Notably, the adoptive transfer of NKT cells into such mice restores tolerance which is dependent on interferon (IFN)-g, IL-10 and/or CXCL16 (Seino et al, Proc Natl Acad Sci USA, 98: 2577-81, 2001; Oh et al, J Immunol, 174: 2030-6, 2005; Jiang et al, J Immunol, 175: 2051-5, 2005; Jiang et al, Am J Transplant, 7: 1482-90, 2007; Ikehara et al, J Clin Invest, 105: 1761-7, 2000). In addition, iNKT cells have proved to be essential for the induction of tolerance to corneal allografts and have been demonstrated to prevent graft-versus-host disease in an IL-4-dependent manner (Sonoda et al, J Immunol, 168: 2028-34, 2002; Zeng et al, J Exp Med, 189: 1073-81 1999; Pillai et al, Blood. 2009; 113:4458-4467; Leveson-Gower et al, Blood, 117: 3220-9, 2011).

iNKT cell responses may depend on the type of transplant carried out, for example, following either vascularized (heart) or non-vascularized (skin) grafts, as the alloantigen drains to iNKT cells residing in the spleen or axillary lymph nodes, respectively. Further, iNKT cell responses can be manipulated, for example, by manipulating iNKT cells to release IL-10 through multiple injection of α-GalCer, which can prolong skin graft survival (Oh et al, J Immunol, 174: 2030-6, 2005).

Achievement of allogeneic immune tolerance while maintaining graft-versus-tumor (GVT) activity has previously remained an elusive goal of allogeneic hematopoietic cell transplantation (HCT). Immune regulatory cell populations including NKT cells and CD4⁺Foxp3⁺ regulatory T (Treg) cells are thought to play a key role in determining tolerance and GVT. To this end, reduced intensity conditioning methods which enrich for NKT and Treg cells have recently been applied with some measure of success. Specifically, a regimen of total lymphoid irradiation (TLI) and anti-thymocyte globulin (ATG) has resulted in engraftment and protection from graft-versus-host disease (GVHD) in both children and adults (Lowsky et al, The New England Journal of Medicine. 2005, 353:1321-1331; Kohrt et al, Blood. 2009; 114:1099-1109; Kohrt et al, European Journal of Immunology. 2010; 40:1862-1869; Pillai et al, Pediatric Transplantation. 2011; 15:628-634) and GVT appeared to be maintained in adult patients whose disease features rendered them at high risk for relapse (Lowsky et al, The New England Journal of Medicine. 2005, 353:1321-1331; Kohrt et al, Blood. 2009; 114:1099-1109; Kohrt et al, European Journal of Immunology. 2010; 40:1862-1869).

Murine pre-clinical modeling of this regimen showed that GVHD protection is dependent upon the IL-4 secretion and regulatory capacity of iNKT cells, and that these cells regulate GVHD while maintaining GVT (Pillai et al, Journal of Immunology. 2007; 178:6242-6251). Further, iNKT-derived IL-4 results can drive the potent in vivo expansion of regulatory CD4⁺CD25⁺Foxp3⁺ Treg cells, which themselves regulate effector CD8⁺ T cells within the donor to prevent lethal acute GVHD (Pillai et al, Blood. 2009; 113:4458-4467). More recently, the present inventors have shown that iNKT cell-dependent immune deviation results in the development and augmentation of function of regulatory myeloid dendritic cells, which in turn induce the potent in vivo expansion of regulatory CD4⁺CD25⁺Foxp3⁺ Treg cells and further enhance protection from deleterious T cell responses (van der Merwe et al, J. Immunol., 2013; epub Nov. 4, 2013, doi:10.4049/jimmunol.1302191). Thus, another envisioned application of iNKT cells is in the augmentation of DC function (both regulatory and pro-inflammatory), for the modulation of effector immune responses and/or tumor immune vaccination strategies. Further applications also include application of iNKT cells to augment regulatory CD4⁺CD25⁺Foxp3⁺ Treg cell expansion or regulatory function.

In response to infection, the immune system relies upon a complex network of signals through the activation of receptors for pathogen-associated molecular patterns, such as the Toll-like receptors (TLRs), expressed on antigen-presenting cells (APC), consequently promoting antigen-specific T cell responses (Medzhitov & Janeway Jr, Science 296: 298-300, 2002). For example, during such responses, iNKT cells respond through the recognition of microbial-derived lipid antigens, or through APC-derived cytokines following TLR ligation, in combination with and without the presentation of self- or microbial-derived lipids. Bacterial antigens can also directly stimulate iNKT cells when bound to CD1d, acting independently of TLR-mediated activation of APC (Kinjo et al, Nat Immunol, 7: 978-86, 2006; Kinjo et al, Nature, 434:520-5, 2005; Mattner et al, Nature, 434: 525-9, 2005; Wang et al, Proc Natl Acad Sci USA, 107: 1535-40, 2010).

Further, NKT (CD1d−/−) and iNKT (Jα18−/−) cell-deficient mice have been shown to be highly susceptible to influenza compared with wild-type mice (De Santo et al, J Clin Invest, 118: 4036-48, 2008). In this model iNKT cells were found to suppress the expansion of MDSC which were expanded in CD1d and Jα18−/− mice (Id.). Importantly, although the exact mechanism of iNKT cell activation was not determined, the authors suggest that iNKT cells required TCR-CD1d interactions, as the adoptive transfer of iNKT cells to Jα18−/− but not CD1d−/− mice suppressed MDSC expansion following infection with PR8 (De Santo et al, J Clin Invest, 118:4036-48, 2008). Thus another application of iNKT cells is in augmentation of immune responses to pathogens (e.g., bacterial, viral, protozoal, and helminth pathogens).

Finally, iNKT cells have been shown to play a critical role in regulating and/or augmenting the allergic immune response, both through secretion of cytokines and through modulation of other immune subsets including regulatory Foxp3+ cells, APCs, and NK cells (Robinson, J Allergy Clin Immunol., 126(6):1081-91, 2010; Carvalho et al., Parasite Immunol., 28(10):525-34, 2006; Koh et al., Hum Immunol., 71(2):186-91, 2010. This includes evidence in atopic dermatitis models (Simon et al., Allergy, 64(11):1681-4, 2009).

Hence an important application of these cells will be in modulation and alleviation of allergic pathology in the skin and multiple internal organs, including in atopic asthma.

However, a major obstacle to application of human innate regulatory iNKT cells in immunotherapy is their relative scarcity in common cellular therapy cell products including human peripheral blood (Berzins et al, Nature Reviews Immunology. 2011; 11:131-142; Exley et al, Current Protocols in Immunology, 2010; Chapter 14: Unit 14-11; Exley & Nakayama, Clinical Immunology, 2011; 140:117-118) and the lack of clear phenotypic and functional data on ex vivo expanded human iNKT cells to validate the potential application of post-expansion human iNKT cells therapeutically.

Despite the great immunological importance and therapeutic potential of iNKT cells and other NKT cells, the art lacks technologies necessary to efficiently expand and/or modulate the activity of NKT cells ex vivo sufficiently to allow their use in therapeutic purposes.

### SUMMARY OF THE INVENTION

As specified in the Background Section, there is a great need in the art to identify technologies for expanding and/or modulating the activity of NKT cells (including both αβ-T cell receptor and γδ-T cell receptor expressing subsets of innate killer cells) ex vivo and to use these technologies to develop novel therapeutics for the treatment and prevention of certain conditions including, e.g., cancer, autoimmunity, inflammatory disorders, tissue transplant-related disorders, infection prevention, and allergic conditions. The present invention satisfies this and other needs.

In one aspect, the invention provides a method for expanding natural killer T (NKT) cells ex vivo, said method comprising the steps of:
(a) harvesting cells from a subject, wherein the cells are selected from the group consisting of peripheral blood mononuclear cells (PBMCs), bone marrow cells, umbilical cord blood cells, and cells of Wharton's jelly;
(b) stimulating cells harvested in step (a) with (i) a glycolipid or a CD1 reagent, (ii) IL-2, and (iii) IL-7;
(c) purifying the resulting stimulated NKT cells, and/or any subset of CD3⁺γδ-TCR⁺ T cells to at least 50% purity by flow cytometry or a magnetic particle-based enrichment procedure;
(d) expanding the NKT cells purified in step (c) in the presence of (i) autologous or allogeneic PBMC feeder cells, (ii) anti-CD3 antibody or anti-TCR-Vα24⁺ antibody, and (iii) IL-2 and/or IL-7, and
(e) optionally re-stimulating the NKT cells expanded in step (d) in the presence of IL-2 and IL-7, and optionally IL-15.

In one embodiment of the above method, the cells are harvested from a subject in step (a) and are introduced back into the same or a different subject after step (d) or (e). In one specific embodiment, the cells are introduced back by a method selected from the group consisting of intravascular infusion, topical application, and irrigation. In one specific embodiment, the recipient subject has a disease selected from the group consisting of cancer, precancerous condition, autoimmune disease, inflammatory condition, transplant rejection, post-transplant lymphoproliferative disorder, allergic disorder, and infection.

In conjunction with the above method for expanding natural killer T (NKT) cells ex vivo, the invention also provides NKT cells produced by said method as well as pharmaceutical compositions comprising such NKT cells and a pharmaceutically acceptable carrier or excipient (e.g., dimethylsulfoxide). In one specific embodiment, such NKT cells are selected from the group consisting of CD3⁺Vα24⁺ iNKT cells, CD3⁺Vα24^{neg} iNKT cells, CD3⁺Vα24^{neg} CD56⁺ NKT cells, CD3⁺Vα24^{neg}CD161⁺ NKT cells, CD3⁺γδ-TCR⁺ T cells, and mixtures thereof.

In another aspect, the invention provides a method of induction of allo-transplant tolerance in a recipient subject in need thereof, said method comprising the steps of:
(a) harvesting cells from the same or a different subject, wherein the cells are selected from the group consisting of peripheral blood mononuclear cells (PBMCs), bone marrow cells, umbilical cord blood cells, and cells of Wharton's jelly;
(b) stimulating cells harvested in step (a) with (i) a glycolipid or a CD1 reagent, (ii) IL-2, and (iii) IL-7;
(c) purifying the resulting stimulated NKT cells, and/or any subset of CD3⁺γδ-TCR⁺ T cells to at least 50% purity by flow cytometry or a magnetic particle-based enrichment procedure;
(d) expanding the NKT cells purified in step (c) in the presence of (i) autologous or allogeneic PBMC feeder cells, (ii) anti-CD3 antibody or anti-TCR-Vα24⁺ antibody, and (iii) IL-2 and/or IL-7;
(e) optionally re-stimulating the NKT cells expanded in step (d) in the presence of IL-2 and IL-7, and optionally IL-15, and
(f) introducing the NKT cells into the recipient subject after step (d) or (e).

In yet another aspect, the invention provides a method of anti-tumor immunotherapy in a recipient subject in need thereof, said method comprising the steps of:
(a) harvesting cells from the same or a different subject, wherein the cells are selected from the group consisting of peripheral blood mononuclear cells (PBMCs), bone marrow cells, umbilical cord blood cells, and cells of Wharton's jelly;
(b) stimulating cells harvested in step (a) with (i) a glycolipid or a CD1 reagent, (ii) IL-2, and (iii) IL-7;
(c) purifying the resulting stimulated NKT cells, and/or any subset of CD3⁺γδ-TCR⁺ T cells to at least 50% purity by flow cytometry or a magnetic particle-based enrichment procedure;
(d) expanding the NKT cells purified in step (c) in the presence of (i) autologous or allogeneic PBMC feeder cells, (ii) anti-CD3 antibody or anti-TCR-Vα24⁺ antibody, and (iii) IL-2 and/or IL-7;
(e) optionally re-stimulating the NKT cells expanded in step (d) in the presence of IL-2 and IL-7, and optionally IL-15, and
(f) introducing the NKT cells into the recipient subject after step (d) or (e).

In a further aspect, the invention provides a method of immune cell therapy in a recipient subject in need thereof, said method comprising the steps of:
(a) harvesting cells from the same or a different subject, wherein the cells are selected from the group consisting of peripheral blood mononuclear cells (PBMCs), bone marrow cells, umbilical cord blood cells, and cells of Wharton's jelly;
(b) stimulating cells harvested in step (a) with (i) a glycolipid or a CD1 reagent, (ii) IL-2, and (iii) IL-7;
(c) purifying the resulting stimulated NKT cells, and/or any subset of CD3⁺γδ-TCR⁺ T cells to at least 50% purity by flow cytometry or a magnetic particle-based enrichment procedure;
(d) expanding the NKT cells purified in step (c) in the presence of (i) autologous or allogeneic PBMC feeder cells, (ii) anti-CD3 antibody or anti-TCR-Vα24⁺ antibody, and (iii) IL-2 and/or IL-7;
(e) optionally re-stimulating the NKT cells expanded in step (d) in the presence of IL-2 and IL-7, and optionally IL-15, and
(f) introducing the NKT cells into the recipient subject after step (d) or (e).

In one embodiment of the latter method, the recipient subject has a disease selected from the group consisting of cancer, precancerous condition, autoimmune disease, inflammatory condition, transplant rejection, post-transplant lymphoproliferative disorder, allergic disorder, and infection.

In one embodiment of any of the above methods of the invention, the cells are introduced into the recipient subject by a method selected from the group consisting of intravascular infusion, topical application, and irrigation.

In one embodiment of any of the above methods of the invention, PBMCs used in step (a) are unmanipulated. In another embodiment of any of the above methods of the invention, PBMCs used in step (a) are pheresed PBMCs. In one embodiment of any of the above methods of the invention, PBMCs used in step (a) have been obtained from an untreated donor. In one embodiment of any of the above methods of the invention, PBMCs used in step (a) have been obtained from a donor mobilized prior to pheresis with a growth factor (e.g., G-CSF) or a chemotherapeutic agent (e.g., cyclophosphamide).

In one specific embodiment of any of the above methods of the invention, the glycolipid in step (b) is α-galactosylceramide (α-GalCer). In another specific embodiment, the glycolipid in step (b) is selected from the group consisting of β-galactosylceramide (β-GalCer), OCH, and PBS-57.

In one embodiment of any of the above methods of the invention, the CD1 reagent in step (b) is a CD1-containing reagent (e.g., CD1d monomer reagents, CD1d dimer, CD1d tetramer, or CD1d multimer). In one embodiment of any of the above methods of the invention, the CD1 reagent in step (b) is selected from the group consisting of ceramide reagents, phospholipids, sphingolipids, phosphatides, sulfatides, phosphonates, and bisphosphonates. In one specific embodiment, the CD1 reagent in step (b) is iNKT-reactive or CD3⁺γδ-TCR⁺ T cell-reactive bisphosphonate (e.g., pamidronate, alendronate, or zoledronic acid/zoledronate).

In one embodiment of any of the above methods of the invention, in step (b) two or more of components (i)-(iii) are used simultaneously or sequentially.

In one embodiment of any of the above methods of the invention, step (b) is conducted for 2 to 14 days. In one specific embodiment, step (b) is conducted for 7 days.

In one embodiment of any of the above methods of the invention, after step (b) is completed, cells are never re-stimulated with a glycolipid or a CD1 reagent.

In one embodiment of any of the above methods of the invention, the resulting stimulated NKT cells in step (c) are selected from the group consisting of CD3⁺Vα24⁺ iNKT cells, CD3⁺Vα24^{neg} iNKT cells, CD3⁺Vα24^{neg}CD56⁺ NKT cells, CD3⁺Vα24^{neg} CD161⁺ NKT cells, CD3⁺γδ-TCR⁺ T cells, and mixtures thereof.

In one embodiment of any of the above methods of the invention, the NKT cells in step (c) are purified by a manual or automated magnetic particle-based enrichment procedure (e.g., manual MACS®, AutoMACS®, CliniMACS®, EasySep®, or RoboSep®).

In one embodiment of any of the above methods of the invention, in step (d) purified NKT cells are expanded for 7 to 35 days.

In one embodiment of any of the above methods of the invention, PBMC feeder cells in step (d) are irradiated PBMC feeder cells. In another embodiment of any of the above methods of the invention, PBMC feeder cells in step (d) are non-irradiated PBMC feeder cells.

In one embodiment of any of the above methods of the invention, step (d) is conducted only once.

In one embodiment of any of the above methods of the invention, step (d)(i) is conducted using allogeneic PBMC feeder cells.

In one embodiment of any of the above methods of the invention, step (d) is conducted without stimulation with a glycolipid or a CD1 reagent.

In one embodiment of any of the above methods of the invention, step (d) is conducted with recurrent stimulation with a glycolipid or a CD1 reagent. In one specific embodiment, the glycolipid in step (d) is α-GalCer. In another specific embodiment, the glycolipid in step (d) is selected from the group consisting of β-GalCer, OCH, and PBS-57. In one specific embodiment, the CD1 reagent in step (d) is a CD1-containing reagent (e.g., CD1d monomer reagents, CD1d dimer, CD1d tetramer, or CD1d multimer). In another specific embodiment, the CD1 reagent in step (d) is selected from the group consisting of ceramide reagents, phospholipids, sphingolipids, phosphatides, sulfatides, phosphonates, and bisphosphonates. In one specific embodiment, the CD1 reagent in step (d) is iNKT-reactive or CD3⁺γδ-TCR⁺ T cell-reactive bisphosphonate (e.g., pamidronate, alendronate, or zoledronic acid/zoledronate). In one specific embodiment, the NKT cell is CD3⁺γδ-TCR⁺ T cell and the CD1 reagent in step (d) is a phosphonate or bisphosphonate compound.

In one embodiment of any of the above methods of the invention, step (e) is conducted for 7-21 days. In one specific embodiment, step (e) is conducted every 7 days for 7-21 days.

In one embodiment of any of the above methods of the invention, the expansion step (d) is conducted in the presence of IL-15.

In one embodiment of any of the above methods of the invention, the feeder cells in the expansion step (d) are PBMC admixed with antigen presenting cells (APCs) expressing 41BBL ligand and IL-15. In one specific embodiment, the feeder cells are PBMC admixed with K-562-41BBL-mIL-15.

In one embodiment of any of the above methods of the invention, the expansion step (d) is conducted in the presence of anti-TCR-Vα24+ antibody.

In one embodiment of any of the above methods of the invention, the purifying in step (c) is conducted using bag culture with enrichment by flow cytometry.

In one embodiment of any of the above methods of the invention, the method further comprises removal of the CD4⁺, CD4⁺, or CD4^{neg}CD8^{neg} subset of NKT cells during the purification step (c).

In one embodiment of any of the above methods of the invention, in step (b) cells are at 2×10⁶ cells/ml and the glycolipid is α-GalCer which is used in concentration 100 ng/ml.

In one embodiment of any of the above methods of the invention, IL-2 and IL-7 are used in steps (b) and (d) at 50-200 U/ml IL-2 and 0.1-400 ng/ml IL-7. In one embodiment of any of the above methods of the invention, IL-2 and IL-7 are used in step (e) at 100 U/ml IL-2 and 0.4 ng/ml IL-7. In one specific embodiment, IL-2 is recombinant human IL-2. In one specific embodiment, IL-7 is recombinant human IL-7.

In one embodiment of any of the above methods of the invention, at least 10⁷ cells are harvested in step (a).

In one embodiment of any of the above methods of the invention, the subject is human. In one embodiment of any of the above methods of the invention, all steps of the method are conducted in a closed-culture system (e.g., a bag system, a bioreactor system, tissue culture apparatus, etc.).

In a separate aspect, the invention provides a method for augmenting cytotoxicity of iNKT cells or CD3⁺γδ-TCR⁺ T cells isolated from a subject, said method comprising activating said iNKT cells or CD3⁺γδ-TCR⁺ T cells with an antibody mixture selected from the group consisting of (i) a mixture of anti-CD2 and anti-CD3 antibodies, (ii) a mixture of anti-CD3 and anti-CD28 antibodies, and (iii) a mixture of anti-CD2, anti-CD3 and anti-CD28 antibodies. In one specific embodiment, the antibody is in a soluble phase. In another specific embodiment, the antibody is loaded to an insoluble or soluble carrier (e.g., beads or a tissue culture surface).

In another aspect, the invention provides a method for augmenting cytotoxicity of iNKT cells or CD3⁺γδ-TCR⁺ T cells isolated from a subject, said method comprising activating said iNKT cells or CD3⁺γδ-TCR⁺ T cells with a reagent capable of activating CD3 complex and/or CD3/CD28 complex signaling in conventional or regulatory T cells. In one embodiment, the reagent capable of activating CD3 complex and/or CD3/CD28 complex signaling in conventional or regulatory T cells is selected from the group consisting of anti-thymocyte serum, anti-thymocyte globulin, anti-CD3 antibodies, globulin containing anti-CD3 antibodies, monoclonally derived anti-CD3 antibodies, and CD3-stimulating compounds.

In a further aspect, the invention provides a method for augmenting cytotoxicity of iNKT cells or CD3⁺γδ-TCR⁺ T cells isolated from a subject, said method comprising activating said iNKT cells or CD3⁺γδ-TCR⁺ T cells with a reagent capable of activating or mimicking signal transduction downstream of the CD3 or CD3/CD28 complex in conventional or regulatory T cells.

In yet another aspect, the invention provides a method for augmenting cytotoxicity of iNKT cells or CD3⁺γδ-TCR⁺ T cells isolated from a subject, said method comprising transducing or transfecting said iNKT cells or CD3⁺γδ-TCR⁺ T cells with a vector capable of activating or mimicking signal transduction downstream of the CD3 or CD3/CD28 complex in conventional or regulatory T cells.

These and other aspects of the present invention will be apparent to those of ordinary skill in the art in the following description, claims and drawings.

### BRIEF DESCRIPTION OF THE DRAWINGS

FIGS. 1A-D show an ex vivo expansion protocol and iNKT immunophenotype. A, Protocol for expansion of iNKT cells. PBMC were stimulated with 100 ng/mL α-GalCer, recombinant human IL-2 and IL-7 for 7 days, at which time CD3⁺Vα24⁺ cells were sorted to >98% purity. Sorted cells were cultured at day 7 with irradiated allogeneic PBMC feeders, stimulation using anti-CD3 antibody, and recombinant human IL-2 and IL-7 weekly for 14-21 days, followed by re-sort and immune phenotyping studies. B, Mean+/−SEM absolute number of CD3⁺Vα24⁺ iNKT cells expanded from human peripheral blood sources (n=49) from 10⁴ starting iNKT cells. Day 0, starting PBMC sample. C, Representative FACS histograms of Vα24 and CD3 on gated CD3+ cells (top row), Vα24 and Vβ11 on gated CD3⁺Vα24⁺ cells (middle row), and CD8 and CD4 staining of gated live CD3⁺Vα24⁺ cells (bottom row), at days 0 (left column), day 7 (middle column), and day 21 (right column) of expansion protocol. D, Representative FACS histograms of CD56 and CD161 staining of gated CD3⁺Vα24⁺CD4⁺ (left), CD3⁺Vα24⁺CD8⁺ (middle) and CD3⁺Vα24⁺CD4^{neg}CD8^{neg} (double-negative, DN) (right) ex vivo expanded iNKT cells at day 21 of expansion protocol. Percentage expression is shown within each quadrant.

FIGS. 2A-D demonstrate regulatory gene expression profile, cytokine secretion, allo-suppressor capacity of ex vivo expanded human peripheral blood iNKT cells. A, Gene expression was measured in iNKT cells from 4 different products. GSEA analyses (top panels) and heat maps (bottom panels) of upregulated pathways (FDR <0.05) in sorted CD3⁺Vα24⁺ iNKT cells at day 28 of expansion, showing expression patterns for NKT genes (first column), inflammatory genes (second column), Th1 and Th2 inflammation (third column), and GATA3 (fourth column). Data represents expression profiles for n=4 separate expansion products on separate donors. B, Mean±SEM cytokine expression (μg/mL) by Luminex® assay (Millipore, Billerica, Calif.) in supernatant of day 28 expanded CD3⁺Vα24⁺ iNKT cells following 24 hours culture without (unstimulated) and with (stimulated) anti-CD2/CD3/CD28 bead stimulation. Data represents mean±SEM of triplicate wells for n=4 separate experiments on separate expansions. (* indicates <50 pg/mL). C, Representative CFSE proliferation histograms of gated CD3⁺CD8⁺ cells at 96 hours when CD3⁺Vα24⁺ iNKT cells were sorted at day 21-28 of expansion and used as suppressors in 96-hour allogeneic MLR assay with allogeneic responders and irradiated third-party allogeneic PBMC stimulators. Percentage of cells in each gate is given above the gate. Results are representative of n=12 total wells each group in n=2 separate experiments, using different donors. (R:S=Responder:Stimulator ratio; +NKT=with addition of CD3⁺Vα24⁺ iNKT cells at a ratio of 1:1 with responders; −NKT=without added iNKT cells). D, Mean proliferation using iNKT cell (day 21) suppressors and T effectors in 72-hr CFSE MLR. R=autologous CD3⁺CD4^{neg}Vα24^{neg} (>95% CD3+CD8+) responders sorted and stored from the original iNKT expansion product; S=irradiated allogeneic PB APC stimulators. (p=0.11 at R: iNKT 1:1 between R:S 1:5 and R:S 1:1).

FIGS. 3A-C show ex vivo expanded NKT cells include a subset of Vα24^{neg} cells (CD3⁺Vα24^{neg} NKT-N cells), which are true NKT cells by gene profiling and functional immunophenotype. A, GSEA analyses (top panels) and heat maps (bottom panels) of upregulated gene expression in sorted CD3⁺Vα24^{neg} NKT-N cells at day 28 of expansion, showing significant activation of pathways (FDR <0.05) for NKT genes (first column), inflammatory genes (second column), Th1 and Th2 inflammation (third column), and GATA3 (fourth column). Data represents expression profiles for n=3 serial expansion products on separate donors. B, Scatter plot of concordant gene expression changes (following stimulation with anti-CD2/CD3/CD28 beads) between CD3⁺Vα24⁺ iNKT cells and CD3⁺Vα24^{neg} NKT-N cells. Axes represent log₂ (fold-change) up-regulation (positive values) and down-regulation (negative values) from 0. Colors represent transcripts significantly altered and shared between iNKT and NKT-N (light grey) as well as non-overlapping gene sets exclusively expressed in iNKT cells (black) or in NKT-N cells (medium grey). FDR: false discovery rate was set at <0.05. Data in A and B represent gene expression profiles for n=4 (iNKT) and n=3 (NKT-N) separate expansion products on separate donors. C, Mean±SEM cytokine expression (pg/mL) by Luminex® assay in supernatant of day 28 expanded CD3⁺Vα24^{neg} NKT-N cells following 24 hours culture without (unstimulated) and with (stimulated) anti-CD2/CD3/CD28 bead stimulation. Data represents mean±SEM of triplicate wells for n=4 separate experiments on separate expansions. (*indicates <50 pg/mL).

FIGS. 4A-G show ex vivo expanded iNKT cells express cytolytic effector molecules and display cytotoxicity against tumor cell targets. A, Representative heat map of InRNA profiling for the top 10 up-regulated genes in overall gene expression profiling of day 28 expanded CD3⁺Vα24⁺ iNKT cells. Gene nomenclature and mean fold-change observed between unstimulated (U) and anti-CD2/CD3/CD28 bead-stimulated (S) samples is given to the right of the heat map. Data represents n=4 separate experiments on separate donors. B, Representative FACS histograms of stimulated Granzyme B in gated CD3⁺Vα24⁺ iNKT cells at day 28. Percentage of cells in each gate is given above the gate. C, Mean+/−SEM percent cytotoxicity as determined by BrightGlo® luciferase assay system (Promega, Madison, Wis.) for expanded CD3⁺Vα24⁺ iNKT cells at day 28 against the B-lymphoblastoid cell lines RS4:11 and Nalm6, and the myeloblastic cell line K562. (E: sorted CD3⁺Vα24⁺ iNKT cell effectors; T: cultured cell line targets). D, Mean+/−SD direct cytotoxicity of TCR-activated day 21 iNKT cells against tumor targets following 6-hour co-incubation of iNKT cells vs control populations with firefly luciferase-transduced (luc⁺) Nalm6 (pre B-ALL) (p<0.01), U937 (monocytic) (p=0.42) and K562 (CML) (p=0.58) targets. (E=iNKT cell effectors; T=targets; p value is at 1:1 E: T ratio vs negative controls). E, Representative mean fluorescence intensity (MFI) of cytolytic effector molecules in fixed and permeabilized expanded PB-iNKT cell (day 21) following 6-hr co-incubation at 1:1 iNKT: target ratio with Nalm6. (Grey histogram=antibody isotype control; Black histogram=GrB/Prf in iNKT sample. GrB=Granzyme B; Prf=Perforin). F, Representative MFI of GrB/Prf in fixed and permeabilized expanded PB-iNKT cell (day 21) following 6-hr co-incubation at 1:1 iNKT: target ratio with RH41 (alveolar rhabdomyosarcoma). (White histogram=antibody isotype control; Grey histogram=GrB/Prf in iNKT sample). G, Representative images (left panel) and mean±SEM cumulative quantitative luminescence (photons/sec) of C.B17 SCID recipients of luc+ NALM/6 xenografts (day 0) followed by infusion of either vehicle (Vehicle) or with day 21 expanded iNKT cells stimulated with α-GalCer, anti-CD2/CD3/CD28 followed by vehicle (CD2/3/28), or with anti-CD2/3/28 and treated with the non-competitive granzyme B inhibitor Z-AAD-CMK for 1 hour prior to infusion (CD2/3/28+CMK). Data represents 18-20 mice per experimental group (n=4 experiments). *, P<0.05; **, P<0.01; ***, P<0.001; NS, non-significant (P>0.05).

FIG. 5 outlines putative mechanisms of iNKT tumor toxicity. Arrow “A” shows how iNKT cells may augment anti-tumor cytolytic capacity of autologous NK cells, via cytokines or contact-dependent augmentation as represented by (++). Arrow “B” shows how iNKT cells may have direct cytotoxicity against tumor targets either via cytokines or via contact-dependent cytolysis. Either “A” or “B” serves as a mechanism of augmentation of cytolytic therapy, particularly after tumor evasion of NK cells (via upregulation of HLA Class I ligands for inhibitory KIR on NK cells) or CD8⁺ T cells (via down-regulation of HLA for Class-I HLA-restricted CD8+ cytolytic T cells) as represented by (−). (KIR=Inhibitory Killer Immunoglobulin-like Receptors).

FIG. 6A provides an alternative optimization of a protocol for NKT expansion using PBMCs supplemented with transduced cell lines as feeders, such feeders including potentially K-562-41BBL-mIL-15 feeders. FIG. 6B provides evidence that use of K-562-41BBL-mIL-15 feeders in such a supplemented expansion protocol can improve the NKT cell yields in expansions (n=10 expansions shown).

### DETAILED DESCRIPTION OF THE INVENTION

As specified in the Background Section, there is a great need in the art to identify technologies for expanding and/or modulating the activity of iNKT cells ex vivo and use this understanding to develop novel therapeutics for the treatment of certain conditions including cancer, autoimmunity, inflammatory disorders, tissue transplant-related disorders and infection. The present invention satisfies this and other needs.

### DEFINITIONS

As used herein in connection with the methods of the invention, the term “natural killer T cell” or “NKT” refers to invariant natural killer T (iNKT) cells as well as all subsets of non-invariant (Vα24^{neg} and Vα24⁺) natural killer T cells which express CD3 and an αβ TCR (herein termed “natural killer αβ T cells”) or γδ TCR (herein termed “natural killer γδ T cells”), all of which have demonstrated capacity to respond to non-protein antigens presented by CD1 antigens. The non-invariant NKT cells encompassed by the methods of the present invention share in common with iNKT cells the expression of surface receptors commonly attributed to natural killer (NK) cells, as well as a TCR of either αβ or γδ TCR gene locus rearrangement/recombination. As used herein, the term “invariant natural killer T cell” or “iNKT” refers to a subset of T-cell receptor (TCR)α—expressing cells which encompasses all subsets of CD3⁺Vα24⁺ iNKT cells (CD3⁺CD4⁺CD8^{neg}Vα24⁺, CD3⁺CD4^{neg} CD8⁺Vα24⁺, and CD3⁺CD4^{neg}CD8^{neg}Vα24⁺) as well as those cells which can be confirmed to be iNKT cells by gene expression or other immune profiling, but have down-regulated surface expression of Vα24 (CD3⁺Vα24^{neg}). This includes cells which either do or do not express the regulatory transcription factor FOXP3.

As used herein, the term “pheresed PBMCs” refers to peripheral blood mononuclear cells (“PBMC”) which have been collected by extracorporeal circulation of blood from donors through an apparatus designed to collect cells at specific sizes, molecular weights, charges, or by addition of specific markers that can be recognized using technologies in or attached to the extracorporeal apparatus (“pheresis”).

The term “CD1 reagent” is used herein to encompass CD1-containing reagents (e.g., CD1 d dimer, tetramer, or other multimer, or CD1d monomer reagents) as well as agents which do not contain CD1 but can be bound by CD1 and presented to NKT cells in CD1 (e.g., ceramide reagents, phospholipids, sphingolipids, phosphatides, sulfatides, phosphonates, and bisphosphonates).

As used in this specification and the appended claims, the singular forms “a,” “an,” and “the” include plural references unless the context clearly dictates otherwise.

The term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

As used herein, the term “subject” refers to any mammal. In a preferred embodiment, the subject is human.

In the context of the present invention insofar as it relates to any of the disease conditions recited herein, the terms “treat”, “treatment”, and the like mean to relieve or alleviate at least one symptom associated with such condition, or to slow or reverse the progression of such condition. Within the meaning of the present invention, the term “treat” also denotes to arrest, delay the onset (i.e., the period prior to clinical manifestation of a disease) and/or reduce the risk of developing or worsening a disease. E.g., in connection with cancer the term “treat” may mean eliminate or reduce a patient's tumor burden, or prevent, delay or inhibit metastasis, etc.

As used herein the term “therapeutically effective” applied to dose or amount refers to that quantity of a compound or pharmaceutical composition that is sufficient to result in a desired activity upon administration to a subject in need thereof. Within the context of the present invention, the term “therapeutically effective” refers to that quantity of a compound or pharmaceutical composition containing such compound that is sufficient to delay the manifestation, arrest the progression, relieve or alleviate at least one symptom of a disorder treated by the methods of the present invention. Note that when a combination of active ingredients is administered the effective amount of the combination may or may not include amounts of each ingredient that would have been effective if administered individually.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A practical Approach, Volumes I and II (D. N. Glover ed. 1985); Oligonucleotide Synthesis (M J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A practical Guide To Molecular Cloning (1984); F. M. Ausubel et al. (eds.), Current Protocols in Molecular Biology, John Wiley & Sons, Inc. (1994); among others.

### Method for Expanding NKT Cells Ex Vivo and Related Compositions

In one aspect, the invention provides a method for expanding natural killer T (NKT) cells ex vivo, said method comprising the steps of:
(a) harvesting cells from a subject, wherein the cells are selected from the group consisting of peripheral blood mononuclear cells (PBMCs), bone marrow cells, umbilical cord blood cells, and cells of Wharton's jelly;
(b) stimulating cells harvested in step (a) with (i) a glycolipid or a CD1 reagent, (ii) IL-2, and (iii) IL-7;
(c) purifying the resulting stimulated NKT cells, and/or any subset of CD3⁺γδ-TCR⁺ T cells to at least 50% purity by flow cytometry or a magnetic particle-based enrichment procedure;
(d) expanding the NKT cells purified in step (c) in the presence of (i) autologous or allogeneic PBMC feeder cells, (ii) anti-CD3 antibody or anti-TCR-Vα24⁺ antibody, and (iii) IL-2 and/or IL-7, and
(e) optionally re-stimulating the NKT cells expanded in step (d) in the presence of IL-2 and IL-7, and optionally IL-15.

In one embodiment of the above method, the cells are harvested from a subject in step (a) and are introduced back into the same or a different subject after step (d) or (e). In one specific embodiment, the cells are introduced back by a method selected from the group consisting of intravascular infusion, topical application, and irrigation. In one specific embodiment, the recipient subject has a disease selected from the group consisting of cancer, precancerous condition, autoimmune disease, inflammatory condition, transplant rejection, post-transplant lymphoproliferative disorder, allergic disorder, and infection.

In conjunction with the above method for expanding natural killer T (NKT) cells ex vivo, the invention also provides NKT cells produced by said method as well as pharmaceutical compositions comprising such NKT cells and a pharmaceutically acceptable carrier or excipient (e.g., dimethylsulfoxide). In one specific embodiment, such NKT cells are selected from the group consisting of CD3⁺Vα24⁺ iNKT cells, CD3⁺Vα24^{neg} iNKT cells, CD3⁺Vα24^{neg}CD56⁺ NKT cells, CD3⁺Vα24^{neg}CD161⁺ NKT cells, CD3⁺γδ-TCR⁺ T cells, and mixtures thereof.

The compositions of the present invention can be used in humans or veterinary animals in therapeutic methods described below or can be administered to a nonhuman mammal for the purposes of obtaining preclinical data. Exemplary nonhuman mammals to be treated include nonhuman primates, dogs, cats, rodents and other mammals in which preclinical studies are performed. Such mammals may be established animal models for a disease to be treated.

### Therapeutic Methods of the Invention

The invention also provides various treatment methods involving delivering NKT cells expanded ex vivo according to the above method of the invention.

In one embodiment, the expanded ex vivo NKT cells are delivered into a subject for treating or preventing cancer or a precancerous condition. In another embodiment, the expanded ex vivo NKT cells are delivered into a subject for treating or preventing diabetes. In another embodiment, the expanded ex vivo NKT cells are delivered into a subject for treating or preventing an inflammatory condition. In another embodiment, the expanded ex vivo NKT cells are delivered into a subject for treating or preventing an autoimmune condition. In another embodiment, the expanded ex vivo NKT cells are delivered into a subject for treating or preventing a transplantation-related condition. In another embodiment, the expanded ex vivo NKT cells are delivered into a subject for treating or preventing graft-versus-host disease. In another embodiment, the expanded ex vivo NKT cells are delivered into a subject for treating or preventing a post-transplant lymphoproliferative disorder. In yet another embodiment, the expanded ex vivo NKT cells are delivered into a subject for treating or preventing an infection.

In one aspect, the invention provides a method of induction of allo-transplant tolerance in a recipient subject in need thereof, said method comprising the steps of:
(a) harvesting cells from the same or a different subject, wherein the cells are selected from the group consisting of peripheral blood mononuclear cells (PBMCs), bone marrow cells, umbilical cord blood cells, and cells of Wharton's jelly;
(b) stimulating cells harvested in step (a) with (i) a glycolipid or a CD1 reagent, (ii) IL-2, and (iii) IL-7;
(c) purifying the resulting stimulated NKT cells, and/or any subset of CD3⁺γδ-TCR⁺ T cells to at least 50% purity by flow cytometry or a magnetic particle-based enrichment procedure;
(d) expanding the NKT cells purified in step (c) in the presence of (i) autologous or allogeneic PBMC feeder cells, (ii) anti-CD3 antibody or anti-TCR-Vα24⁺ antibody, and (iii) IL-2 and/or IL-7;
(e) optionally re-stimulating the NKT cells expanded in step (d) in the presence of IL-2 and IL-7, and optionally IL-15, and
(f) introducing the NKT cells into the recipient subject after step (d) or (e).

In another aspect, the invention provides a method of anti-tumor immunotherapy in a recipient subject in need thereof, said method comprising the steps of:
(a) harvesting cells from the same or a different subject, wherein the cells are selected from the group consisting of peripheral blood mononuclear cells (PBMCs), bone marrow cells, umbilical cord blood cells, and cells of Wharton's jelly;
(b) stimulating cells harvested in step (a) with (i) a glycolipid or a CD1 reagent, (ii) IL-2, and (iii) IL-7;
(c) purifying the resulting stimulated NKT cells, and/or any subset of CD3⁺γδ-TCR⁺ T cells to at least 50% purity by flow cytometry or a magnetic particle-based enrichment procedure;
(d) expanding the NKT cells purified in step (c) in the presence of (i) autologous or allogeneic PBMC feeder cells, (ii) anti-CD3 antibody or anti-TCR-Vα24⁺ antibody, and (iii) IL-2 and/or IL-7;
(e) optionally re-stimulating the NKT cells expanded in step (d) in the presence of IL-2 and IL-7, and optionally IL-15, and
(f) introducing the NKT cells into the recipient subject after step (d) or (e).

In a further aspect, the invention provides a method of immune cell therapy in a recipient subject in need thereof, said method comprising the steps of:
(a) harvesting cells from the same or a different subject, wherein the cells are selected from the group consisting of peripheral blood mononuclear cells (PBMCs), bone marrow cells, umbilical cord blood cells, and cells of Wharton's jelly;
(b) stimulating cells harvested in step (a) with (i) a glycolipid or a CD1 reagent, (ii) IL-2, and (iii) IL-7;
(c) purifying the resulting stimulated NKT cells, and/or any subset of CD3⁺γδ-TCR⁺ T cells to at least 50% purity by flow cytometry or a magnetic particle-based enrichment procedure;
(d) expanding the NKT cells purified in step (c) in the presence of (i) autologous or allogeneic PBMC feeder cells, (ii) anti-CD3 antibody or anti-TCR-Vα24⁺ antibody, and (iii) IL-2 and/or IL-7;
(e) optionally re-stimulating the NKT cells expanded in step (d) in the presence of IL-2 and IL-7, and optionally IL-15, and
(f) introducing the NKT cells into the recipient subject after step (d) or (e).

In one embodiment of the latter method, the recipient subject has a disease selected from the group consisting of cancer, precancerous condition, autoimmune disease, inflammatory condition, transplant rejection, post-transplant lymphoproliferative disorder, allergic disorder, and infection.

Non-limiting examples of cancers treatable by the methods of the invention include, for example, carcinomas, lymphomas, sarcomas, blastomas, and leukemias. Non-limiting specific examples, include, for example, breast cancer, pancreatic cancer, liver cancer, lung cancer, prostate cancer, colon cancer, renal cancer, bladder cancer, head and neck carcinoma, thyroid carcinoma, soft tissue sarcoma, ovarian cancer, primary or metastatic melanoma, squamous cell carcinoma, basal cell carcinoma, brain cancers of all histopathologic types, angiosarcoma, hemangiosarcoma, bone sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, testicular cancer, uterine cancer, cervical cancer, gastrointestinal cancer, mesothelioma, Ewing's tumor, leiomyosarcoma, Ewing's sarcoma, rhabdomyosarcoma, carcinoma of unknown primary (CUP), squamous cell carcinoma, basal cell carcinoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, glioblastoma, astrocytoma, medulloblastoma, craniopharyngioma, ependymoma, pinealoma, hemangioblastoma, acoustic neuroma, oligodendroglioma, meningioma, retinoblastoma, leukemia, neuroblastoma, small cell lung carcinoma, bladder carcinoma, lymphoma, multiple myeloma, medullary carcinoma, B cell lymphoma, T cell lymphoma, NK cell lymphoma, large granular lymphocytic lymphoma or leukemia, gamma-delta T cell lymphoma or gamma-delta T cell leukemia, mantle cell lymphoma, myeloma, leukemia, chronic myeloid leukemia, acute myeloid leukemia, chronic lymphocytic leukemia, acute lymphocytic leukemia, hairy cell leukemia, hematopoietic neoplasias, thymoma, sarcoma, non-Hodgkin's lymphoma, Hodgkin's lymphoma, Epstein-Barr virus (EBV) induced malignancies of all typies including but not limited to EBV-associated Hodkin's and non-Hodgkin's lymphoma, all forms of post-transplant lymphomas including post-transplant lymphoproliferative disorder (PTLD), uterine cancer, renal cell carcinoma, hepatoma, hepatoblastoma, etc.

Non-limiting examples of the inflammatory and autoimmune diseases treatable by the methods of the present invention include, e.g., inflammatory bowel disease (IBD), ulcerative colitis (UC), Crohn's disease, diabetes (e.g., diabetes mellitus type 1), multiple sclerosis, arthritis (e.g., rheumatoid arthritis), Graves' disease, lupus erythematosus, ankylosing spondylitis, psoriasis, Behcet's disease, autistic enterocolitis, Guillain-Barre Syndrome, myasthenia gravis, pemphigus vulgaris, acute disseminated encephalomyelitis (ADEM), transverse myelitis autoimmune cardiomyopathy, Celiac disease, dermatomyositis, Wegener's granulomatosis, allergy, asthma, contact dermatitis, atherosclerosis (or any other inflammatory condition affecting the heart or vascular system), autoimmune uveitis, as well as other autoimmune skin conditions, autoimmune kidney, lung, or liver conditions, autoimmune neuropathies, etc.

Thus, in another embodiment, NKT cells produced by the methods described herein are delivered into a subject for treating or preventing a transplantation-related condition. In another embodiment, NKT cells produced by the methods described herein are delivered into a subject for treating or preventing graft-versus-host disease. In another embodiment, NKT cells produced by the methods described herein are delivered into a subject for treating or preventing a post-transplant lymphoproliferative disorder.

Thus, in yet another embodiment, NKT cells produced by the methods described herein are delivered into a subject for treating or preventing an infection. The infections treatable by the methods of the present invention include, without limitation, any infections (in particular, chronic infections) in which NKT cells are implicated and which can be caused by, for example, a bacterium, parasite, virus, fungus, or protozoa.

It is contemplated that when used to treat various diseases, the compositions and methods of the present invention can be combined with other therapeutic agents suitable for the same or similar diseases. Also, two or more embodiments of the invention may be also co-administered to generate additive or synergistic effects. When co-administered with a second therapeutic agent, the embodiment of the invention and the second therapeutic agent may be simultaneously or sequentially (in any order). Suitable therapeutically effective dosages for each agent may be lowered due to the additive action or synergy.

As a non-limiting example, the invention can be combined with other therapies that block inflammation (e.g., via blockage of IL1, INFα/β, IL6, TNF, IL13, IL23, etc.).

In one embodiment, the compositions and methods disclosed herein are useful to enhance the efficacy of vaccines directed to tumors or infections. Thus the compositions and methods of the invention can be administered to a subject either simultaneously with or before (e.g., 1-30 days before) a reagent (including but not limited to small molecules, antibodies, or cellular reagents) that acts to elicit an immune response (e.g., to treat cancer or an infection) is administered to the subject.

The compositions and methods of the invention can be also administered in combination with an anti-tumor antibody or an antibody directed at a pathogenic antigen or allergen.

The compositions and methods of the invention can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.). The inhibitory treatments of the invention can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e), in any of the aforementioned forms or formulations, alone or in combination with each other or other agents.

Therapeutic methods of the invention can be combined with additional immunotherapies and therapies. For example, when used for treating cancer, NKT cells of the invention can be used in combination with conventional cancer therapies, such as, e.g., surgery, radiotherapy, chemotherapy or combinations thereof, depending on type of the tumor, patient condition, other health issues, and a variety of factors. In certain aspects, other therapeutic agents useful for combination cancer therapy with the inhibitors of the invention include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-receptors, placental proliferin-related protein, as well as those listed by Carmeliet and Jain (2000). In one embodiment, the inhibitors of the invention can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present invention include, for example, aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

For treatment of infections, combined therapy of the invention can encompass co-administering compositions and methods of the invention with an antibiotic, an anti-fungal drug, an anti-viral drug, an anti-parasitic drug, an anti-protozoal drug, or a combination thereof.

Non-limiting examples of useful antibiotics include lincosamides (clindomycin); chloramphenicols; tetracyclines (such as Tetracycline, Chlortetracycline, Demeclocycline, Methacycline, Doxycycline, Minocycline); aminoglycosides (such as Gentamicin, Tobramycin, Netilmicin, Amikacin, Kanamycin, Streptomycin, Neomycin); beta-lactams (such as penicillins, cephalosporins, Imipenem, Aztreonam); vancomycins; bacitracins; macrolides (erythromycins), amphotericins; sulfonamides (such as Sulfanilamide, Sulfamethoxazole, Sulfacetamide, Sulfadiazine, Sulfisoxazole, Sulfacytine, Sulfadoxine, Mafenide, p-Aminobenzoic Acid, Trimethoprim-Sulfamethoxazole); Methenamin; Nitrofurantoin; Phenazopyridine; trimethoprim; rifampicins; metronidazoles; cefazolins; Lincomycin; Spectinomycin; mupirocins; quinolones (such as Nalidixic Acid, Cinoxacin, Norfloxacin, Ciprofloxacin, Perfloxacin, Ofloxacin, Enoxacin, Fleroxacin, Levofloxacin); novobiocins; polymixins; gramicidins; and antipseudomonals (such as Carbenicillin, Carbenicillin Indanyl, Ticarcillin, Azlocillin, Mezlocillin, Piperacillin) or any salts or variants thereof. See also Physician's Desk Reference, 59.sup.th edition, (2005), Thomson P D R, Montvale N.J.; Gennaro et al., Eds. Remington's The Science and Practice of Pharmacy, 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J. Such antibiotics can be obtained commercially, e.g., from Daiichi Sankyo, Inc. (Parsipanny, N.J.), Merck (Whitehouse Station, N.J.), Pfizer (New York, N.Y.), Glaxo Smith Kline (Research Triangle Park, N.C.), Johnson & Johnson (New Brunswick, N.J.), AstraZeneca (Wilmington, Del.), Novartis (East Hanover, N.J.), and Sanofi-Aventis (Bridgewater, N.J.). The antibiotic used will depend on the type of bacterial infection.

Non-limiting examples of useful anti-fungal agents include imidazoles (such as griseofulvin, miconazole, terbinafine, fluconazole, ketoconazole, voriconazole, and itraconizole); polyenes (such as amphotericin B and nystatin); Flucytosines; and candicidin or any salts or variants thereof. See also Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

Non-limiting examples of useful anti-viral drugs include interferon alpha, beta or gamma, didanosine, lamivudine, zanamavir, lopanivir, nelfinavir, efavirenz, indinavir, valacyclovir, zidovudine, amantadine, rimantidine, ribavirin, ganciclovir, foscarnet, and acyclovir or any salts or variants thereof. See also Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

Non-limiting examples of useful anti-parasitic agents include chloroquine, mefloquine, quinine, primaquine, atovaquone, sulfasoxine, and pyrimethamine or any salts or variants thereof. See also Physician's Desk Reference, 59^{th} edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15^{th} edition, (2001), McGraw Hill, NY; Berkow et al., Eds. The Merck Manual of Diagnosis and Therapy, (1992), Merck Research Laboratories, Rahway N.J.

Non-limiting examples of useful anti-protozoal drugs include metronidazole, diloxanide, iodoquinol, trimethoprim, sufamethoxazole, pentamidine, clindamycin, primaquine, pyrimethamine, and sulfadiazine or any salts or variants thereof. See also Physician's Desk Reference, 59^{th} edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15^{th} edition, (2001), McGraw Hill, NY; Berkow et al., Eds. The Merck Manual of Diagnosis and Therapy, (1992), Merck Research Laboratories, Rahway N.J.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

CD1d-restricted iNKT cells are rare but potent innate regulatory cells capable of immune modulation as well as directing anti-tumor cytotoxicity. Protocols to expand iNKT cells and augment their cytotoxicity would allow their application in allogeneic transplantation and anti-tumor immunotherapy. The present example demonstrates ex vivo expansion of highly purified CD3⁺Vα24⁺ iNKT cells from human PBMCs.

This example demonstrates a novel method for ex vivo activation and expansion of human iNKT cells with both alloregulatory and cytotoxic effector function.

This example discloses a method whereby PBMCs were stimulated with the iNKT-specific glycolipid α-GalCer, recombinant IL-2 and IL-7. After sorting to >98% purity on day 7, iNKT cells were further expanded in the presence of irradiated allogeneic PBMCs, anti-CD3 antibody, IL-2 and IL-7, and re-sorted on day 21-28 for immunophenotyping and functional studies. Upon activation, the expanded iNKT cells secreted high levels of both Th1 and Th2 cytokines, GM-CSF, and the chemokines CCL3 and CCL4. They suppressed the proliferation of CD3⁺CD8⁺ effector T cells against allogeneic stimulator cells. Moreover, they unregulated cytolytic effector molecules including granzyme B and exerted cytotoxicity against acute specific tumor cell lines in vitro. This example also demonstrates application of the current invention in producing and/or modulating the activity of iNKT cells and the induction of allogeneic transplant tolerance and anti-cancer immunotherapy.

### Materials and Methods

iNKT Expansions

Peripheral blood apheresis units were obtained from anonymous healthy adult blood donors at St. Jude Children's Research Hospital Blood Donor Center, Memphis, Tenn., under St. Jude Institutional Review Board (IRB) and St. Jude Pathology Department approved protocols. PBMCs were isolated by density-gradient centrifugation using Ficoll-Paque Plus® (GE Healthcare, Piscataway, N.J.). PBMCs at concentration of 2×10⁶ cells/mL were stimulated with 100 ng/mL of the iNKT-specific glycolipid α-GalCer (Funakoshi, Tokyo, Japan), 100 U/mL each of recombinant human IL-2 (Aldesleukin®, Novartis, New York, N.Y.) and rhIL-7 (Sigma-Aldrich, St. Louis, Mo.) for 7 days, after which either CD3⁺Vα24⁺(“+CD4” expansions) or CD3⁺CD4^{neg}Vα24⁺ (“− CD4” expansions) iNKT cells were sorted to >98% purity. Sorted iNKT cells were further expanded in the presence of irradiated (5000 cGy) allogeneic PBMCs, in culture medium containing 1 μg/mL anti-CD3 MoAb (Ancell, Bayport, MN), 100 U/mL rhIL-2 and 0.4-4 ng/mL rhIL-7 in RPMI1640® medium (Cellgro, Manassas, Va.) containing 10 mM HEPES (Thermo Scientific HyClone, Logan, Utah), 0.02 mg/mL gentamicin (Grand Island, N.Y.), and 10% human AB serum (Cellgro) for 14-21 days. The cells were restimulated with rhIL-2 and rhIL-7 on a weekly basis. CD3⁺Vα24⁺ cells were sorted from the expansion cultures to >98% purity using a BD FACSAria-II® Cell Sorter (BD Instruments, Santa Clara, Calif.). Absolute numbers of iNKT cells at each time point were calculated by FACS analysis at the time of sort or, for non-sort time points, by derivation from total cell counts using Trypan blue exclusion and FACS analysis percentages of specific CD3⁺Vα24⁺ or CD3⁺Vα24^{neg} cells stained at indicated days.

Antibodies and Flow Cytometry Analysis (FACS).

The following flow cytometry reagents were used: FITC anti-CD3 (clone HIT3a, BD Pharmingen, San Diego, Calif.), PE-Cy7 anti-CD3 (clone 5K7, BD Pharmingen), Biotin anti-Vα24Jα18 TCR (clone 6B11, eBioscience, San Diego, Calif.; Exley et al., Eur. J. Immunol., 38(6):1756-1766, 2008) followed by PerCP-Cy5.5 conjugated streptavidin (eBioscience), PE anti-Vβ11TCR (clone C21, Beckman Coulter, Brea, Calif.), APC anti-CD4 (clone RPA-T4, BD Pharmingen), APC-Cy7 anti-CD4 (clone RPA-T4, BD Pharmingen) eFluor®450 anti-CD8 (clone OKT8, eBioscience), PE anti-granzyme B (clone GB11, BD Pharmingen), PE-Cy7 anti-IFNγ (clone B27, BD Pharmingen), APC anti-IL-4 (clone 8D4-8, eBioscience), APC-Cy7 anti-CD14 (clone MφP9, BD Pharmingen), PE IgG1κ isotype control (clone MOPC-21, BD Pharmingen), PE-Cy7 IgG1κ isotype control (clone MOPC-21, BD Pharmingen), APC IgGlic isotype control (clone P3.6.2.8.1, eBioscience). Live-Dead Aqua® reagent (LDA, Invitrogen, Carlsbad, Calif.) was used for dead cell exclusion in all FACS analyses and sorts.

Intracellular Staining.

Sorted CD3⁺Vα24⁺ iNKT cells were cultured in 96-well round bottom plates (2×10⁵ cells/well) and stimulated with anti-CD2/CD3/CD28 coated beads (T cell Activation/Expansion Kit, Miltenyi Biotec, Auburn, Calif.). Sorted, unstimulated CD3⁺Vα24⁺ iNKT cells were used as controls. Cells were incubated for 10 hours at 37° C. in 5% CO₂. A monensin-containing transport inhibitor (GolgiStop™, BD) was added in the final 5 hours of culture. Cells were harvested and stained with FITC anti-CD3, biotin anti-Vα24Jα18 TCR followed by PerCP-Cy5.5 conjugated streptavidin, APC-Cy7 anti-CD4, eFluor® 450 anti-CD8 antibodies and LDA for 30 minutes at 4° C. Cells were washed followed by fixation and permeabilization using eBioscience Foxp3 fixation/permeabilization concentrate and diluent solutions according to manufacturer's instructions. Permeabilized cells were incubated with either PE anti-granzyme B, PE-Cy7 anti-IFN-γ and APC anti-IL-4, or the respective isotype control antibodies at 4° C. for 30 minutes, washed using 1× permeabilization solution. Data was acquired using a 4-laser LSR-II® flow cytometer (BD Instruments, San Jose Calif.) and analyzed with FlowJo® 9.4.11 software (TreeStar, Ashland, Oreg.).

Gene Expression Profiling by Microarray Analysis.

RNA was prepared from stimulated and non-stimulated cells using the Qiagen RNeasy Micro® kit (Qiagen Inc., Valencia Calif.). Total RNA from approximately 3×10⁵ cells was converted into cDNA using the NuGEN WTA Pico v2® system (NuGEN Technologies Inc., San Carlos Calif.), fragmented and biotin-labeled using the Encore® Biotin module v2 (NuGEN), and hybridized overnight at 45° C. to an Affymetrix GeneChip PrimeView® human gene expression array (Affymetrix Inc., Santa Clara Calif.). After washing and staining, microarrays were scanned using an Affymetrix GeneChip 3000 7G instrument, and gene expression signals summarized using the RMA algorithm (Irizarry et al, Biostatistics, 2003; 4:249-264). Differentially expressed transcripts were identified by ANOVA (Partek Genomics Suite v6.5, Partek Inc., St. Louis Mo.), and false discovery rates (FDR) were estimated by the Benjamini-Hochberg method (Benjamin & Hochberg, JRStatSocB, 1995; 57: 289-300). The FDR threshold was set to <0.05. Gene lists were analyzed for enrichment of gene ontology and canonical pathway terms using the DAVID bioinformatics databases (Huang et al, Nature Protocols, 2009; 4: 44-57). Gene set enrichment analysis (GSEA) using canonical pathways was performed using GSEA v2.06 software downloaded from the Broad Institute ENREF 15 (Subramanian et al, PNAS, 2005; 102: 15545-15550).

Luminex® Cytokine Profiling.

Sorted iNKT cells (1×10⁵ cells/well) were stimulated with anti-CD2/CD3/CD28 beads for 24 hours and the analysis of the cytokine concentration in the supernatant was performed with the bead-based human cytokine/chemokine Milliplex MAP® 26-plex kit (Millipore, Billerica, Mass.) per manufacturer's instructions. Blanks, standards and quality controls were applied in duplicate, and the samples were applied in triplicate. Fluorescence signal was read on a Multiplex-xMap apparatus (Millipore).

CFSE MLR Suppression Assay.

Responder cells were CD3⁺CD8⁺CD25^{neg} cells sorted from individual apheresis unit-derived PBMCs and labeled with 1 μM 5-,6-carboxy-fluorescein succinimidyl ester (CFSE) (Invitrogen) according to manufacturer's instructions. Stimulator cells were allogeneic PBMCs pre-irradiated on day of MLR at 5000 cGy. CD3⁺Vα24⁺ iNKT cells were sorted at day 21-28 of expansion culture and used as suppressors in the MLR. CD3⁺CD8⁺ responder cells (2.5×10⁴) were cultured in triplicate wells either alone, with stimulators in 1:1 or 1:5 ratio responders:stimulators (R:S), or with stimulators and iNKT cells in 1:1:1, 1:1:5, 1:5:1, or 1:5:5 ratio responders:stimulators:suppressors (R:S:Supp), in a 5% CO₂ incubator at 37° C. At 96 hours after culture, cells were harvested and labeled for CD3, CD4, Vα24Jα18TCR, Vβ11TCR as well as CD19, CD11c and CD14 markers (used to exclude stimulators in data analysis) and analyzed on a 4-laser LSR-II flow cytometer (BD Instruments). Voltage threshold for CFSE at time of FACS analysis was defined using CFSE-labeled responder cells cultured alone for 96 hours. Proliferation in each sample set was measured using the proliferation calculation function of FlowJo® 9.4.11 software (Treestar).

In vitro cytotoxicity assays. Cytotoxic activity of ex vivo expanded CD3⁺Vα24⁺ iNKT cells was assessed using the BrightGlo® luciferase assay system (Promega, Madison, Wis.). Firefly luciferase-transduced (luc+) K562 (ATCC no. CCL-243), R54:11 (ATCC no. CRL-1873) and Nalm6 (DSMZ no. ACC-128) cell lines were maintained in RPMI1640 media supplemented with 10% fetal bovine serum (FBS) (Thermo Scientific HyClone) and used in assays of cytotoxicity as indicated. Luc+K562, RS4:11, and Nalm6 cells (Fujisaki et al, Cancer research, 2009; 69: 4010-4017) were used as targets at 1×10⁵ per well (96-well U-bottom tissue culture plate). Sorted CD3⁺Vα24⁺ iNKT cells stimulated with 1 μg/ml of anti-iNKT antibody (MACS Miltenyi Biotec, Auburn, Calif.) for 12 hours were used as cytotoxic effectors with luc+ target cells. Effectors (E) were incubated with luc+ targets (T) at E:T ratios of 0:1, 0.5:1, 1:1, and 2:1). Each ratio was run in triplicate. Effectors and targets were co-incubated for 4 hours in 37° C. and 5% of CO₂. 100 μL of Bright-Glo (Promega) was added into each well and fluorescence signal was read on a Promega GloMax®-Multi Single-Tube Multimode Reader. Targets alone in analyte medium and analyte medium alone served as controls for background spontaneous lysis and background chemi-luminescence readout, respectively. All background controls gave <1% background lysis in these assays. Similar assays were performed using rhabdomyosarcoma cell line Rh30 and NALM/6 leukemia (American Type Culture Collection/ATCC, Manassas, Va.), using post-co-culture PKH-26 labeling of targets and flow cytometric assessment of cell death by AnnexinV and 7-AAD staining.

Bioluminescence Imaging (BLI).

Tumor xenografts were developed with the St. Jude Xenograft Facility and bioluminescent imaging was performed in collaboration with the St. Jude Live Animal Imaging Core Facility. All mice were monitored, handled, and humanely euthanized in accordance with protocols approved and reviewed annually by the St. Jude Institutional Animal Care and Use Committee (IACUC). The firefly luciferase-transduced (luc+) NALM/6 tumor cell line (courtesy Dr. Dario Campana, Singapore University) was maintained in RPMI-1640 supplemented with 10% Hyclone™ fetal bovine serum (FBS) (Thermo Scientific, Waltham, Mass., USA). Luc+ NALM/6 cells were injected intraperitoneally (i.p.) into 12-week-old male C.B-17 SCID (C.B-Igh-1b/IcrTac-Prkdc^{scid}, Taconic Farm Inc., Hudson, N.Y., USA) (2×10⁵ cells/mouse) (day 0). iNKT cells were stimulated with anti-CD2/CD3/CD28 (Miltenyi Biotec) per manufacturer's instructions for 6 hours and subsequently injected i.v. (day 4) into NALM/6 xenograft-bearing C.B-17 SCID mice. In specific experiments, post-expanded iNKT cells were stimulated with anti-CD2/CD3/CD28 (Miltenyi Biotec) per manufacturer's instructions and then incubated for 1 hour with 1 μM Z-AAD-CMK (ENZO Life Science Inc.), before injection. Vehicle control mice were given sterile PBS (day 4). Mice were randomly assigned to treatment groups before the first imaging (day 7). All mice showing detectable bioluminescent signal at day 21 were included in the analysis (95% of xenografts), as per pre-established criteria. Mice were imaged on days 4 and weekly from day 7 to day 49 using a Xenogen IVIS-200® system (Caliper Life Science, Hopkinton, Mass., USA). Bioluminescence images were acquired 5 minutes after i.p. administration of D-luciferin (SIGMA-Aldrich, Boston, Mass.) (15 mg/mL delivered at 0.1 mL/10 gm body mass) and analyzed using Living Image® Software (version 4.3.1) (Xenogen Corporation, Alameda, Calif., USA). Imaging personnel were blinded to all treatments until conclusion of all data analysis. Bioluminescent signal was quantitated as Total Flux (photons/second) based on a Region of Interest (ROI) encompassing each individual subject in the field of view.

Statistical Analysis.

Statistical significance in differences between mean weekly absolute numbers, mean percentage proliferating cell populations, cytokine concentrations, and photons/sec emission in BLI experiments was analyzed by Mann-Whitney U test using Prism® version 5.0 (GraphPad Software, Inc., La Jolla, Calif.). Statistical analysis for gene expression profiling studies is provided separately. Cytotoxicity assay comparisons were made by 2-way ANOVA on Prism® 5.0 software. For all tests, p<0.05 was considered significant.

### Results

Expansion of Highly Pure CD4⁺, CD8⁺, and CD4^{neg}CD8^{neg} CD3⁺Vα24⁺ Human iNKT Cells.

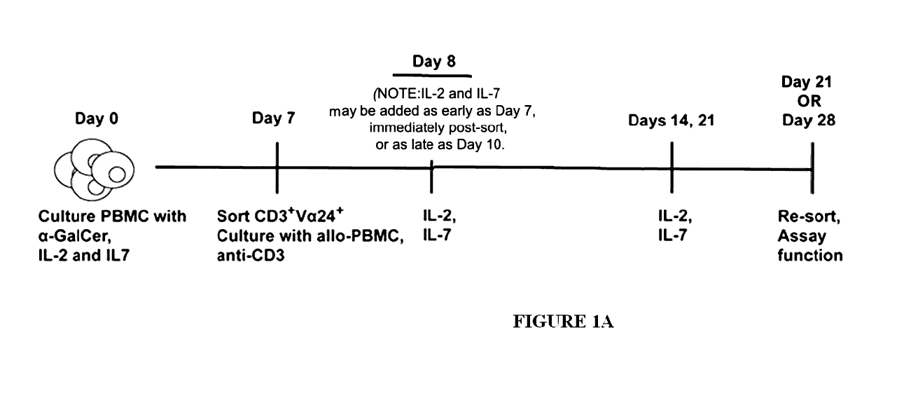

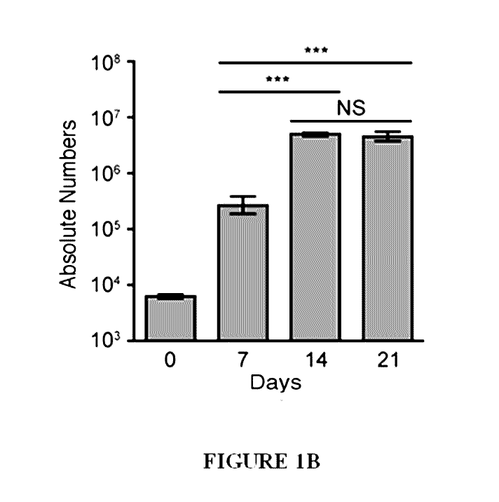

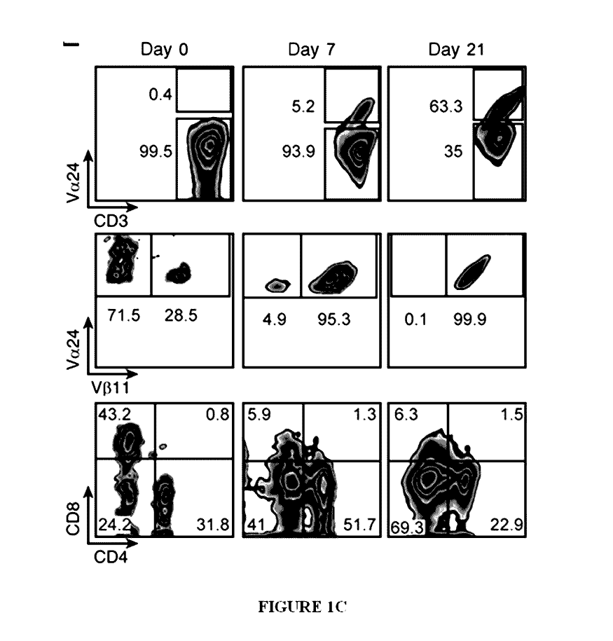

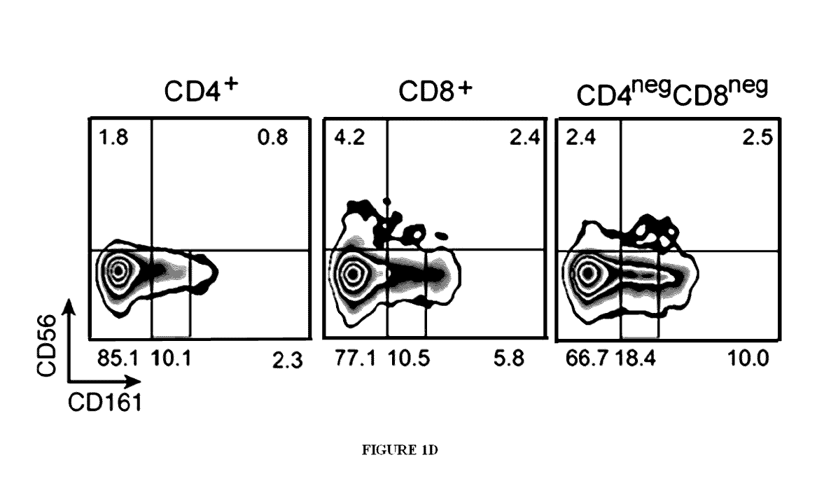

Key elements of the expansion protocol are shown in FIG. 1A. Absolute number CD3+Vα24+ cells per 2×10⁸ starting PBMC (mean+/−absolute numbers for n=49 separate apheresis expansions) is presented in FIG. 1B. FIG. 1C demonstrates representative FACS histograms of Vα24 versus CD3 staining on gated CD3+ cells (top row), and Vα24 versus Vα11 staining (middle row) and CD4 versus CD8 staining (bottom row), respectively, on gated CD3⁺Vα24⁺ cells at initiation of culture (day 0), at day 7 (prior to first sort), and at day 21. As expected, the starting percentage of CD3⁺Vα24⁺ iNKT cells in human blood was very small and ranges from 0.01-1% (FIG. 1C). Greater than 98% of sorted CD3⁺Vα24⁺ cells at days 7 and 28 expressed Vα11 (FIG. 1C), a surface immunophenotype highly specific for human iNKT cells (Berzins et al, Nature reviews Immunology, 2011; 11: 131-142). The expanded iNKT cells generated during the time of this study had variable distribution of percentages of CD4⁺, CD8⁺, and CD4^{neg}CD8^{neg} “double-negative” or “DN” subsets at day 0. However, all three major subsets (CD4⁺, CD8⁺, and CD4^{neg}CD8^{neg}/“double-negative” or “DN”) of human iNKT cells were consistently expanded using this expansion protocol, with the DN iNKT fraction being generally predominant at day 7 and the CD4⁺ iNKT fraction significantly increased by day 21 (FIG. 1C, representative from n=6 analyses). CD3⁺Vα24⁺ iNKT cells were also stained for the NK cells markers CD56 and CD161. A representative FACS histogram of gated CD4⁺, CD8⁺, and DN iNKT cells expressing these markers is shown in FIG. 1D. No subset (CD4⁺, CD8⁺, or DN) of expanded iNKT cells expressed significant CD56. Conversely, CD161 was present on DN, but less frequently on CD4⁺ and CD8⁺ iNKT at day 21.

Gene Expression Profile of Expanded iNKT Cells.

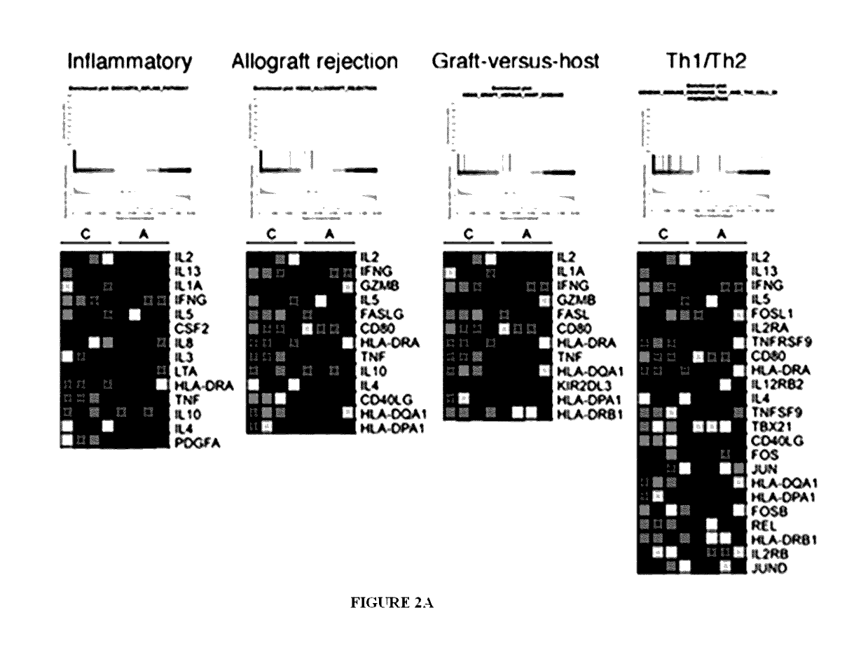

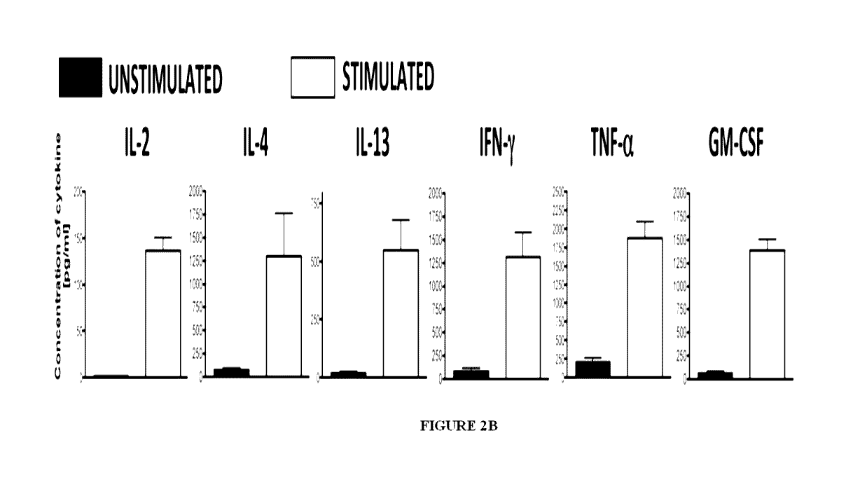

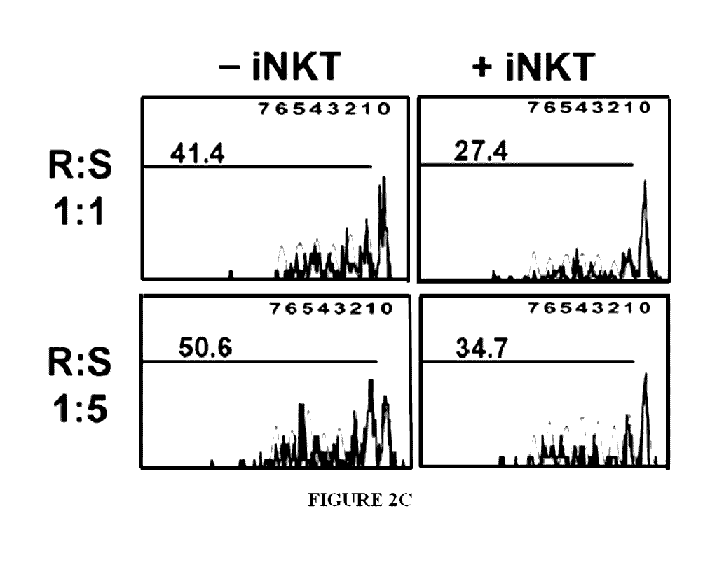

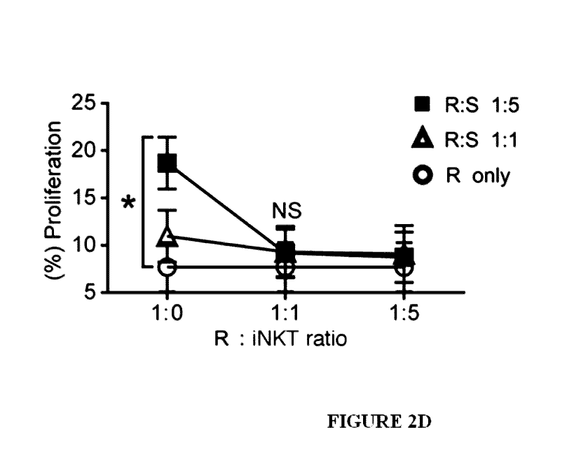

Gene expression was analyzed in day 28 sorted CD3⁺Vα24⁺ iNKT cells from expansion cultures on 4 different randomly selected donors. Gene set enrichment analysis (FIG. 2A) identified significant activation of NKT pathways, with IL2, IL5 and IFNG highly upregulated, inflammatory pathways with high expression of IL2, IL13, IL5 and IFNG. In addition to cell cycle and cellular growth pathways, Th1 and Th2 pathways and GATA3 pathway were also significantly upregulated, with significant expression of IL2 and IFNG (Th1 and Th2), IL13, IL5 and IL4 (GATA3) upregulated in stimulated CD3⁺Vα24⁺ iNKT cells (FIG. 2A).

Cytokine Profile of Ex Vivo Expanded and Anti-CD2/CD3/CD28-Activated iNKT Cells.

A major characteristic of iNKT cells is their capacity to produce both Th1 and Th2 cytokines (Rogers et al, Journal of Immunological Methods, 2004; 285: 197-214; Matsuda et al, Current Opinion in Immunology, 2008; 20: 358-368; Exley et al, The Journal of Experimental Medicine, 1997; 186: 109-120). To determine whether ex vivo expanded CD3⁺Vα24⁺ iNKT cells retained this capacity, the culture supernatants of expanded cells were analyzed for cytokine expression using the MILLIPLEX Map® 26-plex cytokine assay kit after CD3⁺Vα24⁺ iNKT sorted at day 22-28 were stimulated with anti-CD2/CD3/CD28 beads for 24 hours. FIG. 2B shows that stimulated CD3⁺Vα24⁺ iNKT cells expressed nanomolar amounts of IL-4, IFN-γ, TNF-α, CCL3, CCL4, and GM-CSF. The iNKT cells also expressed 0.5 nM IL-13 and very little IL-2 (<0.14 ng/mL). These data show that expanded CD3⁺Vα24⁺, upon stimulation through CD2/CD3/CD28 signaling pathways, release Th1 and Th2 cytokines as well as GM-CSF. These results are in accordance with the canonical cytokine profile reported for iNKT cells.

Ex Vivo Expanded iNKT Cells Display Alloregulatory Capacity In Vitro.

FIG. 2C shows representative CFSE proliferation plots of gated CD3⁺CD8⁺ responders at 96 hours cumulative Mixed Leukocyte Reaction (MLR). Notably, co-culture of day 21-28 expanded iNKT cells with CD3⁺CD8⁺ responders against allogeneic third-party whole PBMC stimulators resulted in significant suppression of proliferation at both 1:1 and 1:5 responder:stimulator (R:S) ratios when iNKT cells expanded from a unit autologous to the responders were added to wells at a ratio of 1:1 responders: suppressors (p<0.01 for R:S 1:1; p<0.001 for R:S 1:5, comparing +iNKT to −iNKT data sets for each R:S ratio) (FIG. 2D). MLR suppression assays using day 21-28 expanded iNKT cells as suppressors and sorted CD3⁺CD4⁺CD25^{neg} responder cells with allogeneic irradiated third-party stimulators produced similar results. This data shows that ex vivo expanded regulatory iNKT cells have capacity to regulate the allo-response between unrelated donor T cells and recipient-type APC, even with potentially significant allo-response barriers, and even when the iNKT cells are derived from an apheresis unit disparate from both responder and stimulator sources.

Characterization of a Subset of Expanded CD3⁺Vα24^{neg} Cells (iNKT-N).

At day 22-28 in iNKT expansion cultures, a persistent population of a CD3⁺Vα24^{neg} was present (FIG. 1C, top row, right column, day 21) despite >98% pure sorting of CD3⁺Vα24⁺ cells at day 7, confirmed by radiation death of all PBMC feeder cells added at day 7. Hereinafter, these cells are referred to as iNKT(Vα24)-Negative (“NKT-N”). The phenotype of these cells was determined. CD3⁺Vα24^{neg} cells were sorted to >98% purity at day 28 and cultured in either medium alone or medium with anti-CD2/CD3/CD28 beads for 24 hours.

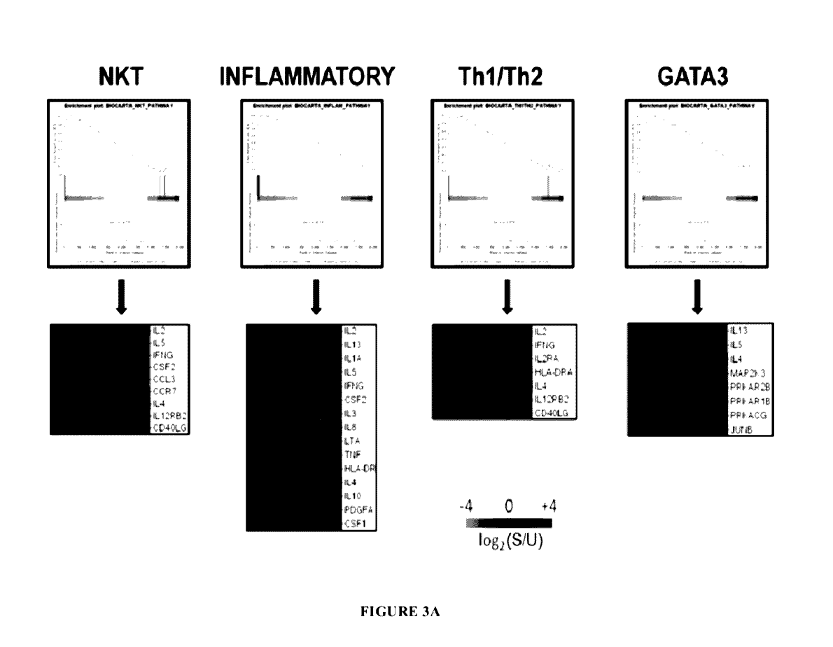

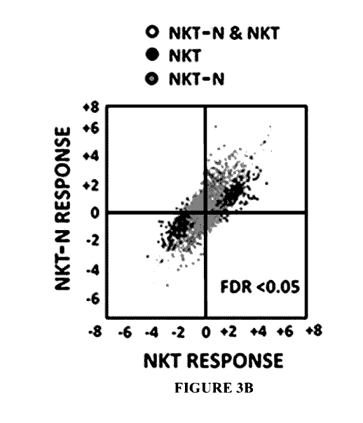

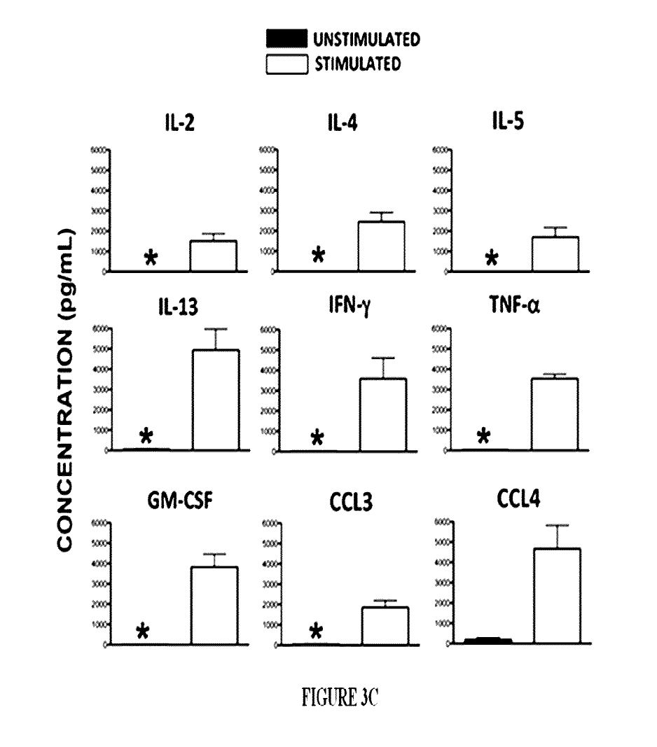

CD3⁺Vα24^{neg} (NKT-N) cells at day 22-28 maintain the overall gene expression profile of iNKT cells. Similar to the analyses in CD3⁺Vα24⁺ cells, gene expression was measured in CD3 Vα24^{neg} NKT-N cells sorted at day 28 from expansion cultures of 4 different randomly selected donors. The global gene expression profile of unstimulated purified CD3 Vα24^{neg} NKT-N cells was compared with that of purified CD3 Vα24^{neg} NKT-N cells stimulated for 24 hours with anti-CD2/CD3/CD28 beads. Gene set enrichment analysis (GSEA) (FIG. 3A) identified significant activation of NKT pathways, with CSF2, CCL3, IFNG and IL-5 highly upregulated, and inflammatory pathways, with high expression of IL1A, IL13, CSF2, IFNG and IL5. In addition to cell cycle and growth pathways, gene sets of Th1 and Th2 pathways and GATA3 pathway were also significantly upregulated, with significant expression of IFNG and IL2RA (Th1 and Th2), IL13, IL5 and IL4 (GATA3) upregulated in stimulated CD3 Vα24^{neg} iNKT cells (FIG. 3D). A scatter plot of concordant gene expression changes following stimulation with anti-CD2/CD3/CD28 beads between CD3⁺Vα24 iNKT cells and CD3⁺Vα24^{neg} NKT-N cells (FIG. 3B) demonstrated near-linear concordance.

Cell culture supernatants were harvested at 24 hours and assayed by MILLIPLEX Map® 26-plex cytokine assay kit. As shown in FIG. 3C, upon stimulation sorted CD3⁺Vα24^{neg} cells secrete an array of cytokines similar to that of CD3⁺Vα24⁺ cells. High levels of IL-13, GM-CSF, IFN-γ, TNF-α and IL-4 were detected in supernatants of bead-stimulated CD3⁺Vα24^{neg} cells compared to unstimulated cells. However, CD3⁺Vα24^{neg} cells released higher amounts of most of the expressed cytokines on a per cell basis when equivalent numbers of CD3⁺Vα24⁺ and CD3⁺Vα24^{neg} cells were used in cytokine assays (p value for comparison of stimulated cytokine values between CD3⁺Vα24^{neg} and CD3⁺Vα24⁺: p=0.0065 for IL-2; p=0.098 for IL-4; p=0.01 for IL-5; p=0.0039 for IL-13; p=0.069 for IFN-γ; p=0.0001 for TNF-α; p=0.0069 for GM-CSF). Stimulated CD3⁺Vα24⁺ cells secreted higher level of CCL3, but not of CCL4, compared to CD3⁺Vα24^{neg} cells (p=0.01 for CCL3 and p=0.1 for CCL4).

The gene expression profile upon activation, surface immunophenotype, and cytokine secretion profile of these CD3⁺Vα24^{neg} cells at day 28 supported their being expanded iNKT cells, which was then confirmed in gene profiling data. Hence, by immunophenotype, cytokine expression signature, and gene expression profiling, CD3⁺Vα24^{neg} NKT-N cells seen at day 22-28 in CD3⁺Vα24⁺ iNKT expansions are a subset of iNKT with downregulated expression of Vα24⁺ and Vα11⁺.

This data support these NKT-N cells may be included in final cell preparations for immunotherapeutic application, and thus allows consideration of final cell preparation via CD3⁺ enrichment (i.e. using CliniMACS® or other enrichment technology) from expansion cultures at day 21-28.

Ex Vivo Expanded iNKT Cells Maintain Cytotoxicity Against Pediatric B-Lymphoblast Cell Lines.

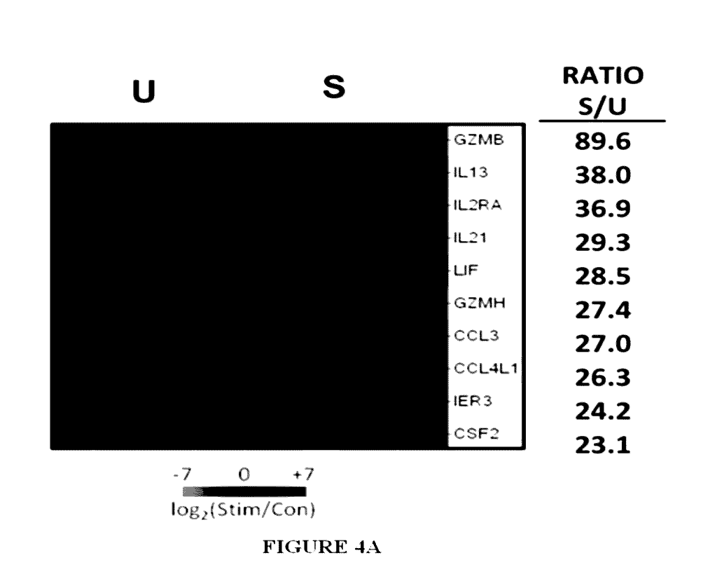

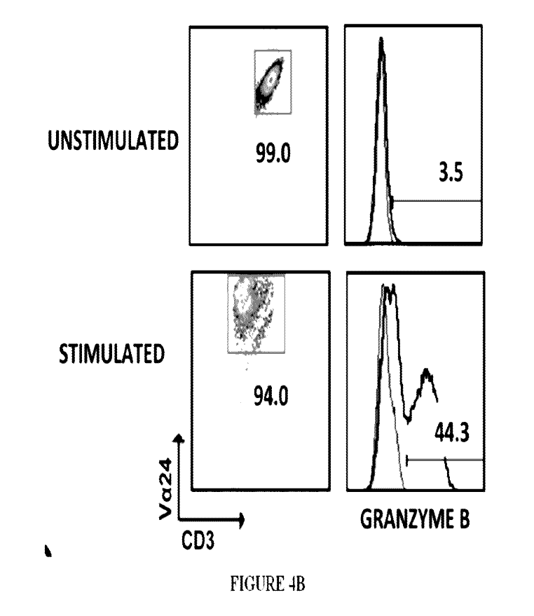

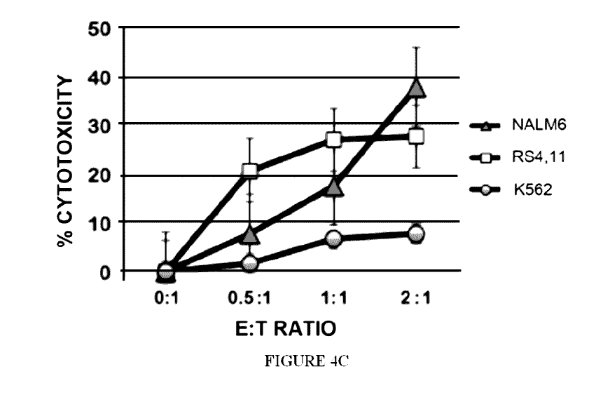

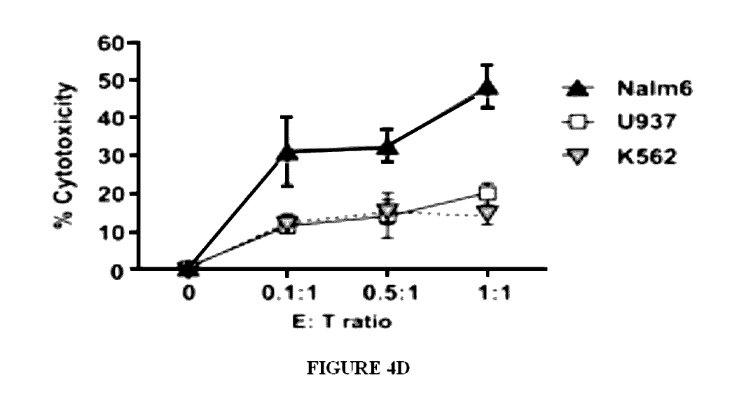

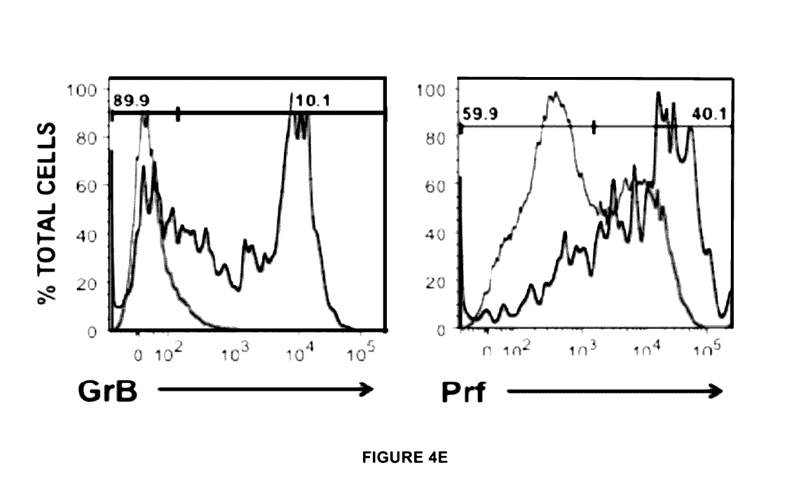

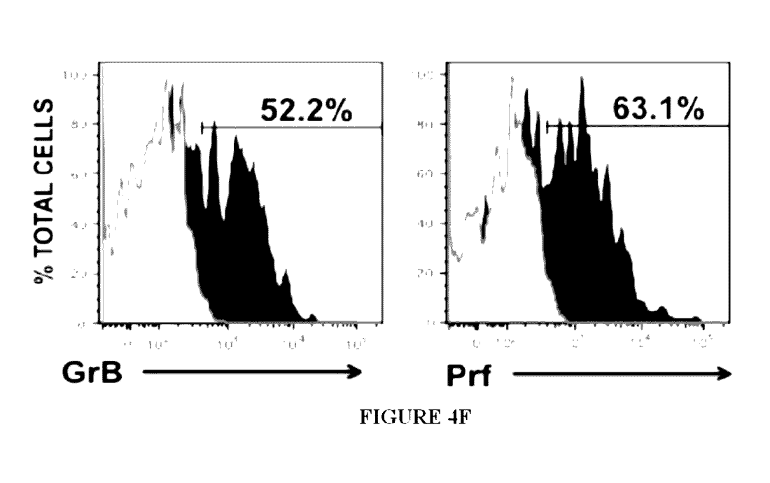

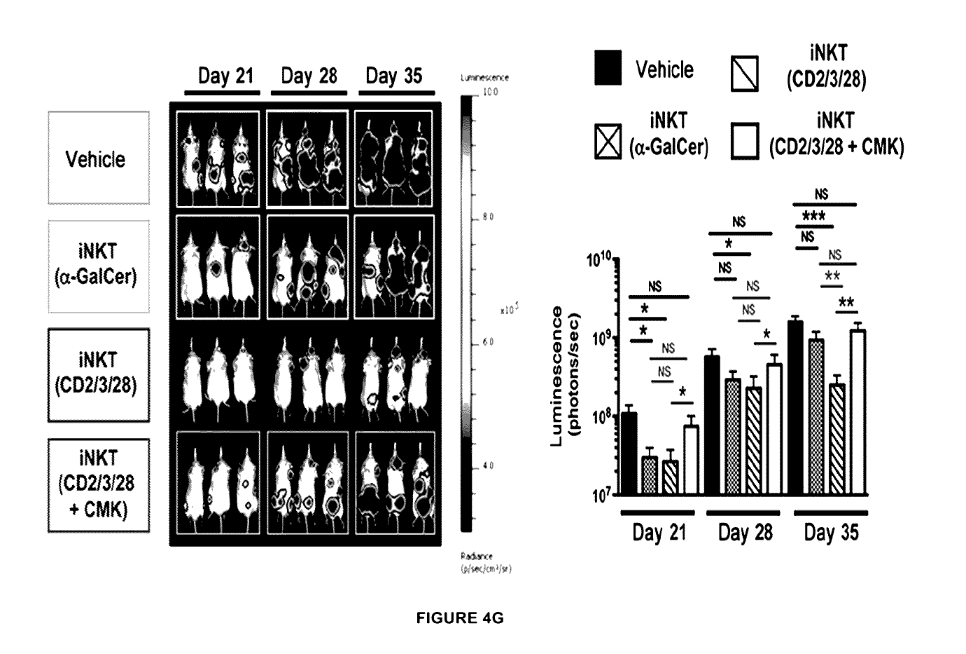

GZMB (Granzyme B) was the most highly upregulated gene (89.6-fold) in CD3⁺Vα24⁺ cells after stimulation with anti-CD2/CD3/CD28 beads compared to unstimulated cells when gene expression was analyzed (FIG. 4A). Granzyme B (GrB) protein expression was confirmed by intracellular staining measured by flow cytometry (FIG. 4B). Similar genes were also upregulated in CD3⁺Vα24^{neg} NKT-N cells, with GZMB again being the most highly up-regulated gene (not shown). There was consistency of upregulated genes, gene expression profiles, and intracellular granzyme B staining across n=4 (iNKT) and n=3 (NKT-N) serial random-donor expansions.

Murine Vα14⁺ iNKT cells exhibit direct cytotoxicity against tumor cells (Cui et al, Science, 1997; 278: 1623-1626) and human Vα24⁺ iNKT cells have demonstrated similar direct cytotoxicity against CD1d-transfected cell lines (Couedel et al, European Journal of Immunology, 1998; 28: 4391-4397; Exley et al, The Journal of Experimental Medicine, 1998; 188: 867-876). Given the expected cytotoxic potential of iNKT cells and the robust and reproducible upregulation of key cytolytic effector molecules including Granzyme B and Granzyme H in iNKT and NKT-N cells following expansion, the direct cytotoxic activity of sorted unactivated CD3⁺Vα24⁺ iNKT cell effectors (E) was examined against B-lineage acute lymphoblastic leukemia cell line targets (T) RS4:11 and Nalm6, and the myeloblastic cell line K562 using the BrightGlo® luciferase assay system (Promega). iNKT cells demonstrated dose-dependent cytotoxicity against B-lymphoid tumor targets Nalm6 cells. As shown in FIG. 4C, 37.7±8.2% (mean±SEM) cytotoxicity was observed at E:T 2:1 ratio, 17.7±7.8% at E:T 1:1, and 7.7±3.8% at E:T 0.5:1 (p values against E:T 0:1 of 0.0004, 0.003, and 0.03, respectively). Direct cytotoxicity was also demonstrated against the B-lymphoblast line RS4:11: 27.7±5.9% at E:T 2:1; 27.1±6.1% at E:T 1:1; 20.8±5.8% at E:T 0.5:1 (p values against E:T 0:1 of 0.002, 0.003, 0.002, respectively). No significant cytotoxicity was observed against the myeloid target K562: 8.8±1.6% at E:T 2:1; 6.7±1.3% at E:T 1:1; 1.8±0.8% at E:T 0.5:1 (p values against E:T 0:1 of 0.10, 0.18, and 0.13, respectively) (FIG. 4C). Results are means of assays done in triplicate from n=3 distinct experiments. Day 21 PB-iNKT cells activated by anti-CD2/CD3/CD28 stimulation exerted potent direct, cell dose-dependent cytotoxicity against B-lineage ALL tumor targets (RS4,11 and Nalm6; Nalm6 shown, FIG. 4D). Potent tumor clearance of established NALM/6 xenografts in C.B17 SCID mice was exerted by directly infused day 21 PB-iNKT cells expanded by the protocol delineated herein was seen when these cells were pre-stimulated using anti-CD2/CD3/CD28 (FIG. 4E). This iNKT tumor clearance capacity was significantly inhibited by pre-blockade of granzyme B in anti-CD2/CD3/CD28 stimulated iNKT cells using the non-competitive/permanent granzyme B-specific inhibitor Z-AAD-CMK (FIG. 4E). Upregulation of Granzyme B (GrB) and Perforin (Prf) are at least two non-exclusive factors that also appear to contribute to the cytotoxicity of ex vivo expanded iNKT cells against B-lymphoid targets (FIG. 4F). Further, GrB/Prf were upregulated in expanded iNKT in contact with alveolar rhabdomyosarcoma (FIG. 4G).

### Discussion

Numerous studies with both murine and human iNKT cells have shown that they are capable of potent immunoregulation, including protection from GVHD and maintenance of GVT or tumor immune surveillance (Pillai et al, Journal of Immunology, 2007; 178: 6242-6251; Hashimoto et al, Journal of Immunology, 2005; 174: 551-556; Morris et al, The Journal of clinical investigation. 2005; 115: 3093-3103; Dellabona et al, Clinical Immunology, 2011; 140: 152-159; de Lalla et al, Journal of Immunology, 2011; 186: 4490-4499; Saito et al, Journal of Immunology, 2010; 185: 2099-2105). However, due to difficulties in expanding them in sufficient numbers ex vivo, the art has lacked the technology necessary to utilize their full regulatory and cytotoxic potential.

This example demonstrates a method to expand iNKT cells ex vivo, with consistent phenotypes of the expanded iNKT cells, achieved using a preliminary phase of specific antigenic stimulation of the iNKT T-cell receptor (TCR) with α-GalCer, followed by a non-antigen specific expansion using CD3 stimulation, allogeneic PBMC feeder cells, and exogenous cytokine support with IL-2 and IL-7 without recurrent stimulation with α-GalCer. This method reliably expands iNKT cells from a limited starting number of total PBMC (range 1.0-5.0×10⁸ starting PBMC in each expansion at day 0). As standard peripheral blood apheresis units often contain 10 to 100-fold higher numbers of total PBMC than these starting PBMC numbers, a very robust yield of highly purified iNKT cells can be produced by this method. In addition, this method can be used to produce iNKT cell of similar yields using closed-culture systems, allowing a direct translational application (e.g., using bag culture, with CliniMACS® enrichment in place of cytometric sorting).

As these day 7 sort yields (FIG. 1B) represent a minimum 10-fold expansion from that beginning at day 0 (FIG. 1B), the total expansion capacity of this protocol is estimated between 500-fold and 5000-fold expansion from calculated primary numbers of iNKT cells at day 0. Further optimizations can increase this yield by 10-fold further and achieve consistent levels appropriate for clinical application.

Three phenotypic subsets within expanded iNKT cells were observed: CD4⁺, CD8⁺, and DN (see also O'Reilly et al, PloS One, 2011; 6:e28648; Rogers et al, Journal of Immunological Methods, 2004; 285: 197-214; Gumperz et al, The Journal of Experimental Medicine, 2002; 195: 625-636). All three of these human iNKT subsets consistently expanded using this protocol, despite the inter-donor variability seen in these iNKT subsets amongst normal blood donors at day 0 of expansion. Notably, CD4⁺ and DN populations are the predominant subtypes expanded. Phenotypic analysis of ex vivo expanded CD4⁺, CD8⁺, and DN iNKT cells demonstrated that expanded DN iNKT cells more frequently express CD161 as compared to CD4⁺ and CD8⁺ subsets, which is in agreement with previous data (O'Reilly et al, PloS One, 2011; 6:e28648; Gumperz et al, The Journal of Experimental Medicine, 2002; 195: 625-636). Significant CD56 expression in expanded iNKT cells produced via the present method was not observed, likely due to the effect of non-TCR-dependent expansion in the final 3 weeks of the protocol in the absence of specific glycolipid stimulation.

Post-expansion human iNKT cells demonstrated clinically relevant functions in vitro including cytokine secretion, allo-regulatory capacity, and cytotoxic activity against tumor cell lines. Though cytokine secretion of ex vivo cultured human iNKT cells has previously been reported (Exley et al, European journal of immunology, 2008; 38: 1756-1766; Rogers et al, Journal of Immunological Methods, 2004; 285: 197-214; Godfrey et al, Nature Immunology, 2010; 11: 197-206; Nishi et al, Human Immunology, 2000; 61: 357-365; van der Vliet et al, Journal of Immunological Methods, 2001; 247: 61-72; Van Kaer et al, Immunotherapy, 2011; 3: 59-75; Matsuda et al, Current Opinion in Immunology, 2008; 20: 358-368) little has been studied or reported regarding human iNKT cell alloregulatory properties or cytotoxicity in vitro. This represents the first time that the cytokine profile, alloregulatory capacity, and cytotoxicity of post-expansion iNKT cells has been characterized together. Moreover, this represents the first time that the gene expression of ex vivo expanded human iNKT cells has been characterized in regards to key regulatory and cytotoxicity-associated molecules of relevance to clinical application. Both CD3⁺Vα24⁺ and CD3⁺Vα24^{neg} iNKT cells secreted high amounts of IL-4, IFN-γ, IL-13, GM-CSF, and TNF-α as assessed by Luminex assay and confirmed by gene expression profiling.

Previous reports have used intracellular cytokine staining of ex vivo expanded iNKT cells (Gumperz et al, The Journal of Experimental Medicine, 2002; 195: 625-636; Lee et al, The Journal of experimental medicine, 2002; 195: 637-641) or assays for secreted cytokines (Rogers et al, Journal of Immunological Methods, 2004; 285: 197-214). A person skilled in the art will appreciate that further optimization of the expansion protocol demonstrated here will result in specific cytokine and chemokine secretion profiles according to iNKT cell subset (CD4⁺, CD8⁺, and DN) following expansion, which may be tailored to their specific clinical application.

Expanded iNKT cells maintained a classic CD3+Vα24+ phenotype and remain >80% viable in cell culture through day 45. It is known that the cytokine profile in iNKT cells is critical to their regulatory functions. (Pillai et al, Blood, 2009; 113 (18): 4458-67; Pillai A, George et al, J Immunol, 2007; 178 (10): 6242-51; Lowsky et al, N Engl J Med, 2005; 353, 13: 1321-1331; Pillai et al, Biology of Blood and Marrow Transplantation 2011; 17(2):s214, Abstract #165). The ex vivo expanded human iNKT cells demonstrated here exhibit potent dose-dependent suppressor activity in allogeneic mixed leukocyte reaction (MLR) (FIGS. 2A, 2B), similar to that seen in freshly isolated human iNKT cells.

One of the functions of α-GalCer activated iNKT cells is the killing of leukemic cells lines in vitro (Takahashi et al, Journal of Immunology, 2000; 164: 4458-4464; Kawano et al, Cancer Research, 1999; 59: 5102-5105; Nicol et al, Immunology, 2000; 99: 229-234). In the present study, CD3⁺Vα24⁺ iNKT cells demonstrated dose-dependent cytotoxicity against B-lymphoid Nalm6 and RS4,11 cells, and possibly other tumor targets (FIGS. 4C, 4D, 4E, 4F).

### Example 2

Allo- and tumor antigen-specific graft-versus-tumor activity (GVT) after hematopoietic cell transplantation (HCT) facilitates immunotherapeutic cure of pediatric leukemias. However, application of HCT is limited by toxicities including lethal graft-versus-host disease (GVHD) when CD8+ T cells are used to drive GVT. Immunosuppressive treatments to prevent or treat GVHD, in turn, inhibit GVT. Therefore, at least one major goal within the art of pediatric allo-HCT for malignancies, is to develop technology to separate GVHD from the GVT capacity of an allograft.

Several recent clinical attempts have been made to optimize GVT against pediatric acute lymphobiastic leukemia (ALL) and acute myeloid leukemia (AML) without GVHD using expanded human natural killer (NK) cells to drive GVT (Ruggeri et al Science 2002, 295(5562): 2097-2100; Ruggeri et al Blood 2007, 110:433-440; Triplett et al, Blood 2006, 107(3):1238-9; Rubnitz et al, 2010, Journal of Clinical Oncology 28(6):955-9.) However, at least one concern in application of NK cell therapies is the potential for tumor immune escape via up-regulation of Class I Human Leukocyte Antigen (HLA) ligands, which can bind inhibitory molecules on NK cells and thereby blunt their cytotoxic effector functions.

A novel strategy is demonstrated here, wherein GVT is augmented without GVHD by use of CD1d-restricted invariant NKT (iNKT) or other subsets of NKT cells.

As discussed in the Background section, supra, NKT cells have strong therapeutic potential outside of HCT, in consolidation and/or combined cellular immunotherapy. NKT cells directly regulate GVHD but maintain anti-tumor activity 16-18 after non-myeloablative allo-transplantation. (Pillai et al, Blood, 2009; 113 (18): 4458-67; Pillai et al, J Immunol 2007; 178 (10): 6242-51; Lowsky et al, N Engl J Med 2005; 353, 13: 1321-1331). Methods to expand NKT cells and to tailor their cytokine secretion would allow broader application and novel treatments for pediatric cancer immunotherapy. Moreover, optimizing understanding of the immunobiology of ex vivo expanded human NKT cells allows therapeutic application of NKT cells to facilitate direct anti-tumor therapy, immune reconstitution, and GVHD prevention in pediatric HCT protocols. Previous NKT expansion protocols were hampered by complex culture requirements and suboptimal yields of NKT cells for realistic clinical application. (Watarai et al, Nature Protocols, 2008; 3 (1); 70-78).

This example demonstrates that robust expansion of highly purified CD3+Vα24+ human iNKT cells can be obtained from multiple cell therapy sources including peripheral blood (PB), bone marrow, and cord blood. This method at least facilitates therapeutic uses of NKT cells related to their direct and indirect cytotoxic affects in immunotherapeutic settings, particularly as they relate to pediatric oncology.

Materials and Methods iNKT cells were sorted to >98% purity from peripheral blood (PB) (hereinafter “PB-iNKT”) following 7-day expansion in the presence of autologous PBMC as a source of antigen-presenting cells (APC) expressing the required iNKT ligand, CD1d. This protocol used Vα24-specific T cell receptor (TCR) stimulation without added glycolipid ligands, and low dose recombinant IL-2 and IL-7. This resulted in mean >10³ fold expansion, with cytolytic effector function against pediatric B-ALL targets. (Pillai et al, Biology of Blood and Marrow Transplantation 2011; 17(2):s214, Abstract #165). This method was further optimized in the protocols outlined in FIG. 1A by use of the known iNKT activating glycolipid ligand alpha-galactosylceramide (α-GalCer) and optimization of cytokine dosage. In one envisioned potential optimization, a transduced K562 cell line (hereinafter “K-562-41BBL-mIL-15 cells”; see Imai et al, Blood 2005; 106(1): 376-383) which expresses 41BBL (a type 2 transmembrane glycoprotein of the TNF-receptor superfamily which binds CD137, a TCR costimulatory receptor which enhances proliferation, survival, and cytolytic function in effector T cells) and membrane bound IL-15 (a common-gamma (−γ) chain cytokine which maintains the viability and augments the cytolytic effector function of expanded NK cells (Fujisaki et al, Cancer Res. 2009; 69(9):4010-7) as the APC feeders to which the iNKT are exposed).

The K-562-41BBL-mIL-15 cell line was created by transfecting the K562 cell line to express 41BBL, a type 2 transmembrane glycoprotein of the TNF-receptor superfamily which binds CD137, a TCR costimulatory receptor which enhances proliferation, survival, and cytolytic function in effector T cells (Imai et al, Blood 2005; 106(1): 376-383; Fujisaki et al, Cancer Res. 2009; 69(9):4010-7).

### Results

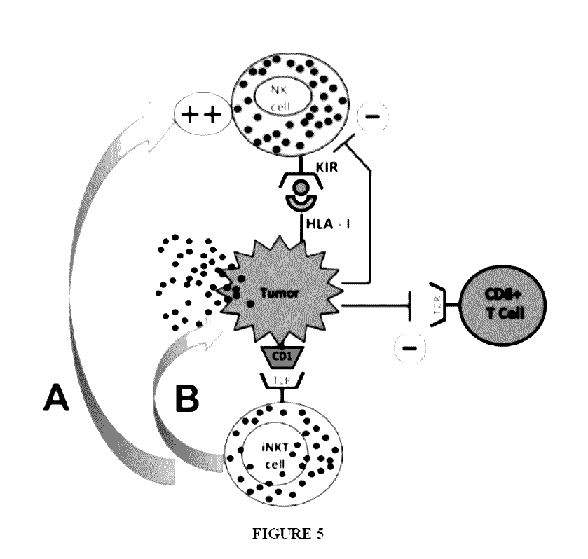

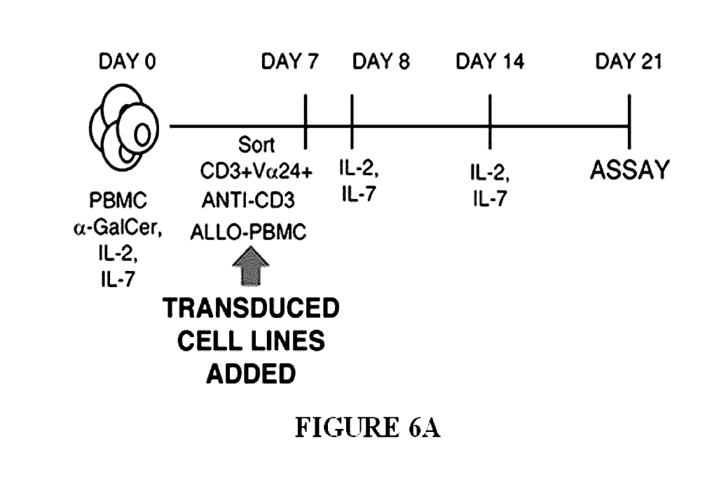

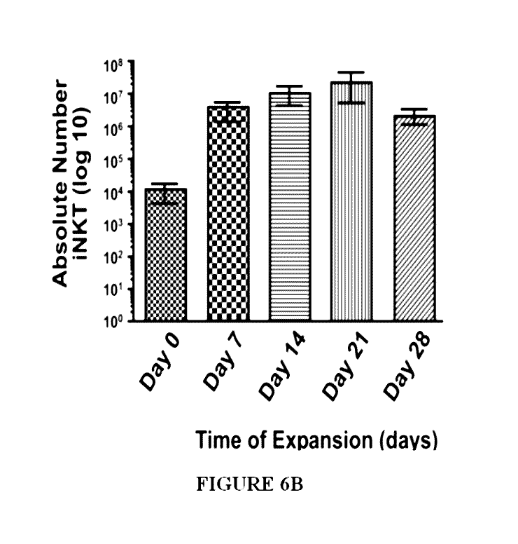

Expression of CD137 on PB-iNKT cells and expression of membrane-bound IL-15 (mIL-15) on K-562-41BBL-mIL-15 cells was confirmed by standard methods. The modified protocol including a transduced cell line in the feeders is outlined in FIG. 6A. Inventor's preliminary data support that CD137 cross-linking combined with mIL-15 augments this iNKT expansion protocol, facilitating >10³-fold expansion and more dependable cell yields (range 3×10⁶−7×10⁷ iNKT cells from 1×10⁴ starting iNKT cells, in 2-3×10⁸ starting PBMC) (FIG. 6B). This stability of expansion yield, and Good Manufacturing Practices (GMP) compatibility of the new APC feeder, further optimizes this protocol for clinical trials of safety and efficacy.

These results indicate that human iNKT cells expressing significant levels of critical regulatory cytokines can be potently expanded within 14-21 days, and these iNKT cells manifest significant direct cytotoxicity against pediatric B-ALL and other high-risk pediatric tumors. These results at least demonstrate significant potential for application of expanded iNKT cells in the pre- or post-transplant setting in immunotherapy of relapsed or high-risk pediatric malignancies.

This data shows iNKT cells possess utility for treating high-risk tumors which express CD1d. As with NK cells, iNKT cells provide options for pretransplant immunotherapy as an alternative to the toxicity of HCT for consolidation. This also has particular application for autologous settings such as treatment of neuroblastoma and rhabdomyosarcoma, where strategies are needed to replace auto-HCT toxicity with directed immunotherapy. Simultaneous expansion of NK and iNKT cells from a single cellular therapy source to augment immunotherapy and prevent tumor escape of high-risk or relapsed pediatric malignancies would allow the paradigm of immunotherapy to move away from HCT toward targeted immunotherapy using synergistic cytolytic iNKT+NK cell therapy.

### Example 3

### Cytotoxicity of Ex Vivo Expanded Human NKT Cells

The cytotoxicity of ex vivo expanded NKT cells (both iNKT and gamma-delta subset NKT) produced according to methods described herein is characterized against pediatric B- and T-ALL, AML, neuroblastoma, alveolar rhabdomyosarcoma, osteosarcoma, and medulloblastoma targets.

### Materials and Methods

NKT cells (both iNKT and gamma-delta subset NKT) are obtained from human peripheral blood pheresis units by modifications to Luszczek et al, Biology of Blood and Marrow Transplantation 2011; 17(2):s214, Abstract #165. (Modified protocol is outlined in FIG. 6A). Ficoll-isolated PBMC are exposed to 100 ng/mL α-GalCer for 7 days, and CD3+CD4^{neg}Vα24+(NKT) cells sorted to >98% purity using FACSAriaII®. NKT cells are stimulated with TCR-Vα24+-specific antibody (Ancell, Bayport, MN), recombinant human IL-2 and IL-7, and K-562-41BBL-mIL-15 feeders.

Day 21 and day 28 NKT cytotoxicity is assessed by 6-hour cytotoxicity with CellTiter-glo® assays (Promega Biosystems, San Luis Obispo, Calif.) in a luciferin-loaded plate with firefly luciferase transduced (luc+) targets. For luc− targets, dual assays with Cytolux® LDH Release Kit (Roche, Indianapolis, Ind.) and DELFIA BATDA® assays are used (PerkinElmer, Waltham, Mass.), and then stained for CD107a and CD107b extrusion on effectors. (Imai et al, Blood, 2005; 106(1): 376-383). Luc+ targets are pre-B-ALL (R54,11; Nalm6), T-ALL (Jurkat, MOLT4), T-lymphoblastic lymphoma (CCRF/CEM) and N-myc amplified and non-amplified neuroblastoma (NB-45D, NBEbC1, CHLA, NBEB, SKNJH), with U937 and K562 negative control cells.

Additional targets which are luciferase negative include alveolar rhabdomyosarcoma (RH41, RH30), chemotherapy-non-responsive osteosarcomas (SAO5, and SJSA-1) and a medulloblastoma line (DAOY). Expression of the NKT ligand CD1d is confirmed on these cells. In vivo tumor kinetic studies are conducted using NKT cells transferred into SCID mice harboring xenografts of the relevant tumor targets. (N=40-60 mice, 2 experimental repeats; N=5 mice per experiment using xenograft+ NKT cells, N=5 controls mice receiving xenograft+vehicle only; N=3-5 mice given NKT only; maximum 2-3 tumor xenografts).

NKT cells' direct cytotoxicity against human B-ALL and T-ALL/lymphoma as well as myeloid targets is determined in 6-hour luciferase and fluorimetric LDH/BATDA assays. NKT cells' cytotoxicity against neuroblastoma and alveolar rhabdomyosarcoma targets, as well as NB-45D and RH41 targets is determined in FACS-based cytotoxicity assays.

### Example 4

NKT (both iNKT and gamma-delta subset NKT) cell targeting can be achieved following expansion by transduction or transfection with specific targeting receptors including but not limited to chimeric antigen receptors (CARs). For example, NKT targeting to B cells or B-cell derived malignancies and cytotoxicity are optimized by expression of the costimulatory signal anti-CD19 chimeric TCR/4-1BB/CD3ζ (anti-CD19-BB-ζ) fusion product on the surface of NKT cells (Imai et al., Blood, 2005; 106(1):376-383). Experiments as described above using B-ALL targets are performed to compare the cytotoxicity of day 21 expanded NKT with and without retroviral transduction using a murine stem-cell virus-internal ribosome entry site-green fluorescent protein (MSCVIRES-GFP) retroviral construct containing a cassette for a surface anti-CD19 chimeric TCR/4-1BB/CD3ζ (anti-CD19-BB-ζ) fusion product (Imai et al., Blood, 2005; 106(1):376-383). This should yield a high percentage of chimeric TCR-GFP expression (Imai et al., Blood, 2005; 106(1):376-383).

NKT cells from N=10-15 human PB pheresis units are expanded as described above, and then transduced with the anti-CD19-BB-ζ vector described above. NKT cells are transduced by stimulation with phytohemagglutinin (7 mg/ml) and IL-2 (200 IU/ml) for 48 h, resuspension in 2-3 mL vector supernatant in RetroNectin (50 μg/mL; TaKaRa, Otsu, Japan) and Polybrene (4 μg/mL; SIGMA) for 2 hours and then re-stimulated with K562-41BBL-mIL-15.

Cytotoxicity is augmented following transduction with anti-CD19-BB-ζ compared to anti-CD19-ζ alone, enhancing cytotoxicity of NKT cells against CD19-expressing B-ALL (RS4,11, Nalm6) targets versus negative control effectors. (Anti-CD19-ζ-truncated serves as a negative control and is equivalent to non-transduced NKT in cytotoxic effector function against B-ALL.)

### Example 5

This example tests whether ex vivo expanded NKT cells (both iNKT and gamma-delta subset NKT) enhance the anti-tumor cytotoxicity of autologous NK cells expanded from the same cellular product source. NKT cells (CD3⁺Vα24⁺) are expanded in parallel with human NK cells (CD3^{neg}Vα24^{neg}CD16⁺CD56⁺) from the same cell therapy product as they can be FACS-sorted without overlap. NK expansion method using K-562-41BBL-mIL-15 feeders is performed as previously described (Imai et al, Blood, 2005; 106(1):376-383). This specific feeder cell line has been tested by the present inventors and shown to generate NKT cells with augmented capacity for cytokine secretion that is anti-inflammatory. Some of these cytokines are capable of augmenting or sustaining the killing response of NK cells. The cytotoxicity is examined of day 21 expanded NKT cells and autologous NK cells co-cultured in direct contact or separated by a cytokine-permeable contact barrier (Transwell® assay) (Life Technologies, Grand Island, N.Y.) with and without addition of blocking monoclonal antibodies to key NKT-derived cytokines including IL-10. Either the NKT side or the NK side of the membrane-separated co-culture is incubated in direct contact with tumor targets as described in examples above. It is determined whether NKT cells augment the ability of NK cells to lyse their tumor targets, in a non-contact dependent manner, via IL-1022 and IFN-γ secretion.

### Example 6

Mechanisms of cytotoxicity of NKT (both iNKT and gamma-delta subset NKT) cells and mechanisms of NKT-mediated augmentation of NK cytotoxicity against pediatric tumor targets are examined using Affymetrix GeneChip® microarray and qRT-PCR, phospho-STAT, and cytokine profiling. Micro-array studies (Mocellin et al, Genes and Immunity, 2004; 5: 621-630) are performed using AffymetrixGeneChip-HT® arrays and GeneTitan® processing. Thresholds for significance are set at 3-4 fold minimum differences in gene expression profile between control samples incubated without targets and samples co-cultured with targets. Expression profiles are also performed for NKT cells incubated with control negative targets (K562 and U937, which are not lysed by NKT cells). Where significant differences exist, expression of relevant cytolytic molecules and profile cytolytic effector pathways in expanded peripheral blood NKT cells is quantified by qRT-PCR. Based on preliminary FACS profiling, it is expected that expanded NKT cells demonstrate upregulation of IL-10, IFN-γ, Signal-Transduction and Activator of Transcription-5 (STATS) pathways, Granzyme B (GrB) and perforin (Prf), and downstream cytolytic effector pathways with lymphoid tumor targets and augmentation of Fas signaling pathways in addition to increased GrB, Prf and STATS pathways following incubation with non-hematolymphoid (solid) tumor targets.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method for expanding natural killer T (NKT) cells ex vivo, said method comprising the steps of:
(a) harvesting cells from a subject, wherein the cells are selected from the group consisting of peripheral blood mononuclear cells (PBMCs), bone marrow cells, umbilical cord blood cells, and cells of Wharton's jelly;
(b) stimulating cells harvested in step (a) with (i) a glycolipid or a CD1 reagent, (ii) IL-2, and (iii) IL-7;
(c) purifying the resulting stimulated NKT cells, and/or any subset of CD3⁺γδ-TCR⁺ T cells to at least 50% purity by flow cytometry or a magnetic particle-based enrichment procedure;
(d) expanding the NKT cells purified in step (c) in the presence of (i) autologous or allogeneic PBMC feeder cells, (ii) anti-CD3 antibody or anti-TCR-Vα24⁺ antibody, and (iii) IL-2 and/or IL-7, and
(e) optionally re-stimulating the NKT cells expanded in step (d) in the presence of IL-2 and IL-7, and optionally IL-15.

2-5. (canceled)

6. The method of claim 1, wherein the CD1 reagent in step (b) is iNKT-reactive or CD3⁺γδ-TCR⁺ T cell-reactive bisphosphonate.

7. The method of claim 1, wherein the glycolipid in step (b) is α-galactosylceramide (α-GalCer).

8. The method of claim 1, wherein the glycolipid in step (b) is selected from the group consisting of β-galactosylceramide (β-GalCer), OCH, and PB S-57.

9-12. (canceled)

13. The method of claim 1, wherein step (b) is conducted for 2 to 14 days.

14-15. (canceled)

16. The method of claim 1, wherein the resulting stimulated NKT cells in step (c) are selected from the group consisting of CD3⁺Vα24⁺ iNKT cells, CD3⁺Vα24^{neg} iNKT cells, CD3⁺Vα24^{neg}CD56⁺ NKT cells, CD3⁺Vα24^{neg}CD161⁺ NKT cells, CD3⁺γδ-TCR⁺ T cells, and mixtures thereof.

17-18. (canceled)

19. The method of claim 1, wherein in step (d) purified NKT cells are expanded for 7 to 35 days.

20-32. (canceled)

33. The method of claim 1, wherein step (e) is conducted for 7-21 days.

34. The method of claim 33, wherein step (e) is conducted every 7 days for 7-21 days.

35. The method of claim 1, wherein the expansion step (d) is conducted in the presence of IL-15.

36. The method of claim 1, wherein the feeder cells in the expansion step (d) are PBMC admixed with antigen presenting cells (APCs) expressing 41BBL ligand and IL-15.

37. The method of claim 36, wherein the feeder cells are PBMC admixed with K-562-41BBL-mIL-15.

38. The method of claim 1, wherein the expansion step (d) is conducted in the presence of anti-TCR-Vα24+ antibody.

39. (canceled)

40. The method of claim 1, further comprising removal of the CD4⁺, CD4⁺, or CD4^{neg}CD8^{neg} subset of NKT cells during the purification step (c).

41-52. (canceled)

53. Natural killer T (NKT) cells produced by the method of claim 1.

54. The NKT cells of claim 53, wherein the cells are selected from the group consisting of CD3⁺Vα24⁺ iNKT cells, CD3⁺Vα24^{neg} iNKT cells, CD3⁺Vα24^{neg}CD56⁺ NKT cells, CD3⁺Vα24^{neg}CD161⁺ NKT cells, CD3⁺γδ-TCR⁺ T cells, and mixtures thereof.

55. A pharmaceutical composition comprising the NKT cells of claim 53 and a pharmaceutically acceptable carrier or excipient.

56. (canceled)

57. A method of induction of allo-transplant tolerance in a recipient subject in need thereof, said method comprising the steps of:
(a) harvesting cells from the same or a different subject, wherein the cells are selected from the group consisting of peripheral blood mononuclear cells (PBMCs), bone marrow cells, umbilical cord blood cells, and cells of Wharton's jelly;
(b) stimulating cells harvested in step (a) with (i) a glycolipid or a CD1 reagent, (ii) IL-2, and (iii) IL-7;
(c) purifying the resulting stimulated NKT cells, and/or any subset of CD3⁺γδ-TCR⁺ T cells to at least 50% purity by flow cytometry or a magnetic particle-based enrichment procedure;
(d) expanding the NKT cells purified in step (c) in the presence of (i) autologous or allogeneic PBMC feeder cells, (ii) anti-CD3 antibody or anti-TCR-Vα24⁺ antibody, and (iii) IL-2 and/or IL-7;
(e) optionally re-stimulating the NKT cells expanded in step (d) in the presence of IL-2 and IL-7, and optionally IL-15, and
(f) introducing the NKT cells into the recipient subject after step (d) or (e).

58. A method of anti-tumor immunotherapy in a recipient subject in need thereof, said method comprising the steps of:
(a) harvesting cells from the same or a different subject, wherein the cells are selected from the group consisting of peripheral blood mononuclear cells (PBMCs), bone marrow cells, umbilical cord blood cells, and cells of Wharton's jelly;
(b) stimulating cells harvested in step (a) with (i) a glycolipid or a CD1 reagent, (ii) IL-2, and (iii) IL-7;
(c) purifying the resulting stimulated NKT cells, and/or any subset of CD3⁺γδ-TCR⁺ T cells to at least 50% purity by flow cytometry or a magnetic particle-based enrichment procedure;
(d) expanding the NKT cells purified in step (c) in the presence of (i) autologous or allogeneic PBMC feeder cells, (ii) anti-CD3 antibody or anti-TCR-Vα24⁺ antibody, and (iii) IL-2 and/or IL-7;
(e) optionally re-stimulating the NKT cells expanded in step (d) in the presence of IL-2 and IL-7, and optionally IL-15, and
(f) introducing the NKT cells into the recipient subject after step (d) or (e).

59. A method of immune cell therapy in a recipient subject in need thereof, said method comprising the steps of:
(a) harvesting cells from the same or a different subject, wherein the cells are selected from the group consisting of peripheral blood mononuclear cells (PBMCs), bone marrow cells, umbilical cord blood cells, and cells of Wharton's jelly;
(b) stimulating cells harvested in step (a) with (i) a glycolipid or a CD1 reagent, (ii) IL-2, and (iii) IL-7;
(c) purifying the resulting stimulated NKT cells, and/or any subset of CD3⁺γδ-TCR⁺ T cells to at least 50% purity by flow cytometry or a magnetic particle-based enrichment procedure;
(d) expanding the NKT cells purified in step (c) in the presence of (i) autologous or allogeneic PBMC feeder cells, (ii) anti-CD3 antibody or anti-TCR-Vα24⁺ antibody, and (iii) IL-2 and/or IL-7;
(e) optionally re-stimulating the NKT cells expanded in step (d) in the presence of IL-2 and IL-7, and optionally IL-15, and
(f) introducing the NKT cells into the recipient subject after step (d) or (e).

60-117. (canceled)
